SEQUENCE LISTING (8 sequences)

SEQ ID NO: 1 - protein, 1124 aa, Homo sapiens
MAPPSEETPLIPQRSCSLLSTEAGALHVLLPARGPGPPQRLSFSFGDHLAEDLCVQAAKASGILPVYHSLFALATEDLSCWFPPSHIFSVEDASTQVLLYRIRFYFPNWFGLEKCHRFGLRKDLASAILDLPVLEHLFAQHRSDLVSGRLPVGLSLKEQGECLSLAVLDLARMAREQAQRPGELLKTVSYKACLPPSLRDLIQGLSFVTRRRIRRTVRRALRRVAACQADRHSLMAKYIMDLERLDPAGAAETFHVGLPGALGGHDGLGLLRVAGDGGIAWTQGEQEVLQPFCDFPEIVDISIKQAPRVGPAGEHRLVTVTRTDNQILEAEFPGLPEALSFVALVDGYFRLTTDSQHFFCKEVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCCIPRPKEKSNLIVVQRGHSPPTSSLVQPQSQYQLSQMTFHKIPADSLEWHENLGHGSFTKIYRGCRHEVVDGEARKTEVLLKVMDAKHKNCMESFLEAASLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFS

SEQ ID NO: 2 - DNA, 4025 bp, Homo sapiens
cccggctctgcccgcccttcgaaagtccagggtccctgcccgctaggcaagttgcactcatggcacctccaagtgaagagacgcccctgatccctcagcgttcatgcagcctcttgtccacggaggctggtgccctgcatgtgctgctgcccgctcggggccccgggcccccccagcgcctatctttctcctttggggaccacttggctgaggacctgtgcgtgcaggctgccaaggccagcggcatcctgcctgtgtaccactccctctttgctctggccacggaggacctgtcctgctggttccccccgagccacatcttctccgtggaggatgccagcacccaagtcctgctgtacaggattcgcttttacttccccaattggtttgggctggagaagtgccaccgcttcgggctacgcaaggatttggccagtgctatccttgacctgccagtcctggagcacctctttgcccagcaccgcagtgacctggtgagtgggcgcctccccgtgggcctcagtctcaaggagcagggtgagtgtctcagcctggccgtgttggacctggcccggatggcgcgagagcaggcccagcggccgggagagctgctgaagactgtcagctacaaggcctgcctacccccaagcctgcgcgacctgatccagggcctgagcttcgtgacgcggaggcgtattcggaggacggtgcgcagagccctgcgccgcgtggccgcctgccaggcagaccggcactcgctcatggccaagtacatcatggacctggagcggctggatccagccggggccgccgagaccttccacgtgggcctccctggggcccttggtggccacgacgggctggggctgctccgcgtggctggtgacggcggcatcgcctggacccagggagaacaggaggtcctccagcccttctgcgactttccagaaatcgtagacattagcatcaagcaggccccgcgcgttggcccggccggagagcaccgcctggtcactgttaccaggacagacaaccagattttagaggccgagttcccagggctgcccgaggctctgtcgttcgtggcgctcgtggacggctacttccggctgaccacggactcccagcacttcttctgcaaggaggtggcaccgccgaggctgctggaggaagtggccgagcagtgccacggccccatcactctggactttgccatcaacaagctcaagactgggggctcacgtcctggctcctatgttctccgccgcagcccccaggactttgacagcttcctcctcactgtctgtgtccagaacccccttggtcctgattataagggctgcctcatccggcgcagccccacaggaaccttccttctggttggcctcagccgaccccacagcagtcttcgagagctcctggcaacctgctgggatggggggctgcacgtagatggggtggcagtgaccctcacttcctgctgtatccccagacccaaagaaaagtccaacctgatcgtggtccagagaggtcacagcccacccacatcatccttggttcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcatggatgccaagcacaagaactgcatggagtcattcctggaagcagcgagcttgatgagccaagtgtcgtaccggcatctcgtgctgctccacggcgtgtgcatggctggagacagcaccatggtgcaggaatttgtacacctgggggccatagacatgtatctgcgaaaacgtggccacctggtgccagccagctggaagctgcaggtggtcaaacagctggcctacgccctcaactatctggaggacaaaggcctgccccatggcaatgtctctgcccggaaggtgctcctggctcgggagggggctgatgggagcccgcccttcatcaagctgagtgaccctggggtcagccccgctgtgttaagcctggagatgctcaccgacaggatcccctgggtggcccccgagtgtctccgggaggcgcagacacttagcttggaagctgacaagtggggcttcggcgccacggtctgggaagtgtttagtggcgtcaccatgcccatcagtgccctggatcctgctaagaaactccaattttatgaggaccggcagcagctgccggcccccaagtggacagagctggccctgctgattcaacagtgcatggcctatgagccggtccagaggccctccttccgagccgtcattcgtgacctcaatagcctcatctcttcagactatgagctcctctcagaccccacacctggtgccctggcacctcgtgatgggctgtggaatggtgcccagctctatgcctgccaagaccccacgatcttcgaggagagacacctcaagtacatctcacagctgggcaagggcaactttggcagcgtggagctgtgccgctatgacccgctaggcgacaatacaggtgccctggtggccgtgaaacagctgcagcacagcgggccagaccagcagagggactttcagcgggagattcagatcctcaaagcactgcacagtgatttcattgtcaagtatcgtggtgtcagctatggcccgggccgccagagcctgcggctggtcatggagtacctgcccagcggctgcttgcgcgacttcctgcagcggcaccgcgcgcgcctcgatgccagccgcctccttctctattcctcgcagatctgcaagggcatggagtacctgggctcccgccgctgcgtgcaccgcgacctggccgcccgaaacatcctcgtggagagcgaggcacacgtcaagatcgctgacttcggcctagctaagctgctgccgcttgacaaagactactacgtggtccgcgagccaggccagagccccattttctggtatgcccccgaatccctctcggacaacatcttctctcgccagtcagacgtctggagcttcggggtcgtcctgtacgagctcttcacctactgcgacaaaagctgcagcccctcggccgagttcctgcggatgatgggatgtgagcgggatgtccccgccctctgccgcctcttggaactgctggaggagggccagaggctgccggcgcctcctgcctgccctgctgaggttcacgagctcatgaagctgtgctgggcccctagcccacaggaccggccatcattcagcgccctgggcccccagctggacatgctgtggagcggaagccgggggtgtgagactcatgccttcactgctcacccagagggcaaacaccactccctgtccttttcatagctcctgcccgcagacctctggattaggtctctgttgactggctgtgtgaccttaggcccggagctgcccctctctgggcctcagaggccttatgagggtcctctacttcaggaacacccccatgacattgcatttgggggggctcccgtggcctgtagaatagcctgtggcctttgcaatttgttaaggttcaagacagatgggcatatgtgtcagtggggctctctgagtcctggcccaaagaagcaaggaaccaaatttaagactctcgcatcttcccaaccccttaagccctggccccctgagtttccttttctgtctctctctttttattttttttatttttatttttatttttgagacagagcctcgctctgttacccagggtggagtgcagtggtgcgatctcggctcagtgcaacctctgcttcccaggttcaagcgattctcctgcctcagcctcccgagtagctgggattacaggtgtgcaccaccacacccggctaattttttttatttttaatagagatgaggtttcaccatgatggccaggctgatctcgaactcctaacctcaagtgatcctcccacc

SEQ ID NO: 3 - DNA, 37 bp, Artificial Sequence
  Synthetic oligonucleotide primer.
gtaaaacgacggccagtggtgttcccctaaggcaggt

SEQ ID NO: 4 - DNA, 38 bp, Artificial Sequence
  Synthetic oligonucleotide primer.
caggaaacagctatgaccgtgttgctggggctgtcata

SEQ ID NO: 5 - DNA, 20 bp, Artificial Sequence
  Synthetic oligonucleotide primer.
cgctaggcgacaatacaggt

SEQ ID NO: 6 - DNA, 27 bp, Artificial Sequence
  Synthetic oligonucleotide primer.
ataattctagacacatatgcccatctg

SEQ ID NO: 7 - DNA, 32 bp, Artificial Sequence
  Synthetic oligonucleotide primer.
gcatggagtcattcctggaagtagcgagcttg

SEQ ID NO: 8 - DNA, 31 bp, Artificial Sequence
  Synthetic oligonucleotide primer.
cttccaggaatgactccatgcagttcttgtg

(19) United States
(12) Patent Application Publication
(10) Pub. No.: US 2009/0285796 A1
(43) Pub. Date: Nov. 19, 2009

(54) MUTANT JAK3 KINASE IN HUMAN LEUKEMIA

(75) Inventors: Valerie Goss, Seabrook, NH (US); Ting-lei Gu, Woburn, MA (US); Roberto Polakiewicz, Lexington, MA (US); Brian Druker, Portland, OR (US); Denise Walters, Rochester, MN (US)

Correspondence Address: KLARQUIST SPARKMAN, LLP, 121 SW SALMON STREET, SUITE 1600, PORTLAND, OR, 97204, US

(73) Assignee: CELL SIGNALING TECHNOLOGY, INC., Danvers, MA (US)

(21) Appl. No.: 12/161,512

(22) PCT Filed: Jan. 19, 2007
(86) PCT No.: PCT/US2007/001359

§ 371 Date: Jun. 3, 2009

Related U.S. Application Data
(60) Provisional application No. 60/760,673, filed on Jan. 20, 2006.

(51) Int. Cl. A61K 39/395; C07H 21/04; C07K 14/00; C12N 15/63; C12N 5/10; C12N 9/12; C07K 16/00; C12Q 1/68; G01N 33/53; A61K 31/7088; A61P 35/02

(52) U.S. Cl. 424/130.1; 536/23.1; 536/23.2; 530/350; 435/320.1; 435/325; 435/194; 530/389.1; 530/391.3; 435/6; 435/7.1; 514/44 A

(57) ABSTRACT

In accordance with the invention, a novel activating mutation (alanine 572 to valine) in JAK3 kinase has been discovered in human acute myelogenous leukemia (AML). The mutant JAK3 kinase was confirmed to drive the proliferation and survival of acute megakaryoblastic leukemia (AML-M7). The invention therefore provides, in part, isolated polynucleotides and vectors encoding the disclosed mutant JAK3 kinase polypeptides, probes for detecting it, isolated mutant polypeptides, recombinant polypeptides, and reagents for detecting the mutant polypeptides. The disclosed identification of this new mutant protein and enables new methods for determining the presence of mutant JAK3 kinase polypeptides in a biological sample, methods for screening for compounds that inhibit the mutant proteins, and methods for inhibiting the progression of a cancer characterized by the mutant polynucleotides or polypeptides, which are also provided by the invention.

### RELATED APPLICATIONS

This application claims priority to and the benefit of U.S. Ser. No. 60/760,673, filed Jan. 20, 2006 (presently pending), the disclosure of which is hereby incorporated herein by reference.

### JOINT RESEARCH AGREEMENT

This application describes and claims certain subject matter that was developed under a written joint collaborative research agreement between CELL SIGNALING TECHNOLOGY, INC., and THE OREGON HEALTH & SCIENCE UNIVERSITY, having an effective date of May 4, 2004, pertaining to markers of cancer and drug resistance.

### FIELD OF THE INVENTION

The invention relates generally to proteins and genes involved in cancer, and to the detection, diagnosis and treatment of cancer.

### BACKGROUND OF THE INVENTION

Many cancers are characterized by disruptions in cellular signaling pathways that lead to aberrant control of cellular processes, or to uncontrolled growth and proliferation of cells. These disruptions are often caused by changes in the activity of particular signaling proteins, such as kinases. Among these cancers are leukemias, which include four major subtypes, acute lymphocytic leukemia, chronic lymphocytic leukemia, acute myelogenous leukemia (AML), and chronic myelogenous leukemia. There are about 35,000 new cases of AML in the United States annually, and it is estimated that almost 23,000 patients will die each year from the disease in the United States alone. See “Cancer Facts and Figures 2005,” American Cancer Society. Among the four subtypes of leukemia, AML, which itself includes various subtypes such as acute megakaryoblastic leukemia (AML-M7), is the most common and most deadly.

It is known that gene translocations resulting in kinase mutant proteins with aberrant signaling activity can directly lead to certain cancers. For example, it has been directly demonstrated that the BCR-ABL oncoprotein, a tyrosine kinase mutant protein, is the causative agent in human chronic myelogenous leukemia (CML). The BCR-ABL oncoprotein, which is found in at least 90-95% of CML cases, is generated by the translocation of gene sequences from the c-ABL protein tyrosine kinase on chromosome 9 into BCR sequences on chromosome 22, producing the so-called Philadelphia chromosome. See, e.g. Kurzock et al., N. Engl. J. Med. 319: 990-998 (1988). The translocation is also observed in acute lymphocytic leukemia and AML cases.

A limited number of gene translocations leading to mutant or mutant proteins implicated in a variety of other hematological cancers have been described. See, e.g., review by Falini et al., Blood 99(2); 409-426 (2002). Among such translocations are the NPM-ALK mutant (involved in anaplastic large cell lymphoma), the E2A-PBX/HLF mutant (involved in B-cell acute lymphoblastic leukemia), and the NPM-MLF1 mutant (involved in myelodysplastic syndrome/AML). See id. In AML, the translocation of OTT-MAL at t(1; 22)(p13; q13) has been described (see Mercher et al., Genes Chromosomes Cancer 33(1): 22-8 (2002)), as has the AML1-MTG8 t(8:21) translocation (see Ohki et al., U.S. Pat. No. 5,580,727 (December 1996)). Mutations within the kinase domain of FLT3 and/or internal tandem duplication of the FLT3 juxtamembrane domain have been detected in over 30% of patients with AML. See Yokota et al., Leukemia 11: 1605-1609 (1997); Yamamoto et al., Blood 97: 2434-2439 (2001). These alterations of FLT3 result in constitutive FLT3 kinase activity and increased cellular proliferation.

Recently, a number of reports have described the presence of an activating mutation (V617F) in the JH2 pseudo-kinase domain of the JAK2 kinase in patients with polycythemia vera, essential thrombo-cythemia, and idiopathic myelofibrosis. See Baxter et al., Lancet 365: 1054-1061 (2005); James et al., Nature 434:1144-1148 (2005); Zhao et al., J. Biol. Chem. 280: 22788-22792 (2005); Levine et al., Cancer Cell 7: 387-97 (2005); Jones et al., Blood [E-publication ahead of print] (2005). The same mutation was also detected in an AML cell line. See Levine, supra. Structural analysis of JAK2 predicted that when JAK2 is in the inactive form its JH1 and JH2 regions interact at their C-terminal ends (critical regions of interaction occurring within residues V617-E621 of JAK2) and prevent protein activation. See Lindauer et al., Protein Eng. 14(1): 27-37 (2001). It has also been demonstrated that the JH2 domain of JAK3 has a similar inhibitory function with regard to protein activation. See Saharinen et al., J. Biol. Chem. 277. 47954-47963 (2002). Several diseases have been reported to have JAK3 activation, including mantle cell lymphoma, Hodgkin disease and colon cancer. See, e.g., Yared et al., Arch. Pathol. Lab. Med. 129(8): 990-6 (2005); Lin et al., Am. J. Pathol. 167(4):969-80 (2005).

Identifying translocations and mutations in human cancers is highly desirable because it can lead to the development of new therapeutics that target such mutant proteins, and to new diagnostics for identifying patients that have such mutations. For example, BCR-ABL has become a target for the development of therapeutics to treat leukemia. Recently, Gleevec® (Imatinib mesylate, STI-571), a small molecule inhibitor of the ABL kinase, has been approved for the treatment of CML. This drug is the first of a new class of anti-proliferative agents designed to interfere with the signaling pathways that drive the growth of tumor cells. The development of this drug represents a significant advance over the conventional therapies for CML and ALL, chemotherapy and radiation, which are plagued by well known side-effects and are often of limited effect since they fail to specifically target the underlying causes of the malignancies. Likewise, reagents and methods for specifically detecting BCR-ABL mutant protein in patients, in order to identify patients most likely to respond to targeted inhibitors like Gleevec® have been described.

Accordingly, there remains a need for the identification of novel mutations, particularly activating mutations in kinases, that result in mutant proteins implicated in the progression of human cancers, including leukemias, and the development of new reagents and methods for the study and detection of such mutant proteins. Identification of such proteins will, among other things, desirably enable new methods for selecting patients for targeted therapies, as well as for the screening of new drugs that inhibit such mutant proteins.

### SUMMARY OF THE INVENTION

In accordance with the invention, a novel activating point mutation (A572V) in Janus Kinase-3 (JAK3) occurring in human myelogenous leukemia (AML) has now been identified. The mutant JAK3 kinase, which has increased tyrosine kinase activity as compared to wild type JAK3, was confirmed to drive the proliferation and survival of a human acute megakaryoblastic leukemia (AML-M7) cell line, CMK.

The invention therefore provides, in part, isolated polynucleotides and vectors encoding the disclosed mutant JAK3 polypeptides, probes and assays for detecting them, isolated mutant JAK3 polypeptides, recombinant mutant polypeptides, and reagents for detecting the mutant JAK3 polynucleotides and polypeptides. The disclosed identification of this new mutant JAK3 kinase protein enables new methods for determining the presence of mutant JAK3 polynucleotides or polypeptides in a biological sample, methods for screening for compounds that inhibit the mutant kinase protein, and methods for inhibiting the progression of a cancer characterized by the expression of mutant JAK3 polynucleotides or polypeptides, which are also provided by the invention. The aspects and embodiments of the invention are described in more detail below.

### BRIEF DESCRIPTION OF THE DRAWINGS

FIGS. 1A-1B—are the amino acid sequence (1 letter code) of human JAK3 kinase (SwissProt Accession No. P52333) (SEQ ID NO: 1) (FIG. 1A) and coding DNA sequence of human JAK3 kinase (GeneBank Accession No. NM_{—}000215) (SEQ ID NO: 2) (FIG. 1B); the underlined residues indicate the JH2 pseudo-kinase domain, the italics indicate the kinase domain, and the residue indicated in bold (alanine 572) is mutated to valine in the disclosed mutant protein.

FIG. 2—is a Western blot analysis of extracts from four human AML cell lines (including CMK) showing constitutive tyrosine phosphorylation of STAT5 (a substrate of JAK3 kinase) in the CMK cell line following serum starvation.

FIG. 3—is a graph (panel A) showing that treatment of a human AML cell line (CMK) with increasing concentrations of pan JAK inhibitor, JAK I, results in a decrease in proliferation (n=3±SD); a bar graph (panel B) indicating that increasing concentrations of the inhibitor JAK I results in an increase in apoptosis of CMK cells but not K562 cells as measured by annexin V staining (n=3±SD); and a Western blot (panel C) indicating that incubation of CMK cells with the inhibitor JAK I results in decreased phosphorylation of STAT5.

FIG. 4—shows the effects of JAK siRNA on the AML cell line, CMK: (panel A) is a Western blot indicating siRNA-induced silencing of JAK family kinases in whole cell lysates of CMK cells 48 hours after siRNA transfection; (panel B) is a bar graph indicating that electroporation with JAK1-specific and JAK3-specific siRNA into CMK cells results in a decrease of proliferation (n=7±SD); (panel C) is a bar graph indicating an increase in apoptosis (n=3±SD) of CMK cells following inhibition of JAK1 and JAK3 expression; and (panel D) is a Western blot showing that siRNA down-regulation of JAK1 and JAK3 results in decreased phosphorylation of STAT5.

FIG. 5—Shows bar graphs indicating that the AML cell line, CMK, demonstrates a greater increase in apoptosis when the cells are treated simultaneously with JAK1 and JAK3 inhibiting siRNA (n=3±SD) (panel A), and that treatment of CMK cells with both JAK1-specific and JAK3-specific siRNA results in a greater decrease in proliferation (n=3±SD) (panel B); and Western blots showing that simultaneous siRNA-induced down regulation of both JAK1 and JAK3 48 hrs after transfection (panel C), JAK1 and JAK3 co-immunoprecipitate in CMK cells (panel 0), and that down-regulation of JAK3 with siRNA decreases phosphorylation of JAK1, but not vice versa (panel E).

FIG. 6—Indicates that transfection of the mutant (A572V) JAK3 kinase confers a growth advantage to Ba/F3 cells: (panel A) is a Western blot showing JAK3, P-STAT5 and STAT5 expression in transfected and untransfected Ba/F3 cells; and (panel B) is a graph showing that the growth of Ba/F3 cells transfected with the mutant JAK3 are substantially more interleukin (IL)-3 independent than Ba/F3 transfected with wild type JAK3, using the MTS assay.

### DETAILED DESCRIPTION OF THE INVENTION

The present invention was made, in part, with grant funding from the U.S. Government, which may have certain rights in the subject matter disclosed and claimed herein.

In accordance with the invention, a previously unknown point mutation (alanine 572 to valine (A572V)) in JAK3 kinase, which results in activation of this kinase, has now been identified in human acute megakaryoblastic leukemia (AML-M7), a subtype of acute myelogenous leukemia (AML). The mutant JAK3 kinase was confirmed to drive the proliferation and survival of a human leukemia cell line, CMK.

Although a limited number of mutations resulting in activated kinases have been described in AML, including the translocation of OTT-MAL at t(1; 22)(p13; q13) (see Mercher et al., supra.), mutations in FLT3 kinase (see Yokota et al., supra.; Yamamoto et al., supra.), and the activating mutation (V617F) in the JH2 domain JAK2 kinase (see Levine et al., supra.), the presently disclosed A572V activating point mutation in JAK3 kinase is novel. JAK3 is non-receptor tyrosine kinase, and member of the JAK family of kinases, which associate with various cytokine receptors lacking intrinsic kinase activity. The protein is highly conserved in mammals, including rats, mice, cats, and pigs. JAK3 phosphorylates, and activates, the STAT family of transcription factors, including STAT5, and other signaling molecules including IRS1/2 and PI3K. It is expressed in cells of hematopoietic and epithelial origins, particularly natural-killer (NK) cells. Defects in JAK3 expression and/or activity have been found in a variety of cancers, including mantle cell lymphoma, Hodgkin disease and colon cancer. See Yared et al., supra.; Lin et al., supra. Although no activating point mutations in JAK3 have previously been described, there is at least one known activating mutation (V617F) in the related kinase, JAK2. See, e.g., Levine et al., supra.

As further described below, the mutant (A572V) JAK3 protein has presently been isolated (wild type JAK3 sequence is published), and a cDNA for expressing the mutant kinase produced. Accordingly, the invention provides, in part, isolated polynucleotides that encode active mutant JAK3 polypeptides, nucleic acid probes that hybridize to such polynucleotides, and methods, vectors, and host cells for utilizing such polynucleotides to produce recombinant mutant JAK3 polypeptides. The invention also provides, in part, isolated polypeptides comprising amino acid sequences encoding active mutant JAK3 polypeptides of the invention, recombinant mutant polypeptides, and isolated reagents that specifically bind to and/or detect mutant JAK3 polypeptides, but do not bind to or detect wild type JAK3. These aspects of the invention, which are described in further detail below, will be useful, inter alia, in further studying the mechanisms of cancers driven by mutant JAK3 kinase expression/activity, for identifying leukemias and other cancers characterized by expression/activity of the JAK3 (A572V) mutant, and in practicing methods of the invention as further described below.

The identification of the novel JAK3 kinase mutant has important implications for the potential diagnosis and treatment of diseases, such as leukemia, that are characterized by this activated mutant protein. Leukemias, for example, are often difficult to diagnose until after they have advanced, increasing the difficulty of effectively treating or curing this disease. AML itself, the most common type of leukemia, is an aggressive disease, with a 5-year survival rate of less than 20%. See American Cancer Society, supra. The acute megakaryoblastic (AML-7) subtype of AML is a rare form of pediatric AML that is most prevalent in young children with Down's Syndrome. See A. Verschurr, Ophanet Encyclopedia (May 2004) (orpha.net/data/patho/GB/uk-AMLM7.pdf. Treatment of AML remains difficult, and the first-line therapy of aggressive multi-drug chemotherapy regimes is hard on patients and associated with significant mortality. Alternatively, total body irradiation coupled with chemotherapy and bone marrow transplant may be employed, which are similarly traumatic for patients.

Therefore, the discovery of the A572V JAK3 kinase mutant, which is presently shown to drive proliferation and survival of a subtype of human AML, enables important new methods for accurately identifying mammalian leukemias (such as AML), as well as other cancers, in which the activated mutant JAK3 kinase is expressed. These tumors are most likely to respond to inhibitors of the kinase activity of the JAK3 mutant protein, such as the pan-JAK inhibitor JAK I (Calbiochem Cat. # 420099). The ability to identify, as early as possible, cancers that are driven by activated mutant JAK3 kinase will greatly assist in clinically determining which therapeutic, or combination of therapeutics, will be most appropriate for a particular patient, thus helping to avoid prescription of inhibitors targeting other kinases that are not in fact, the primary signaling molecule driving the cancer.

Accordingly, the invention provides, in part, methods for detecting the presence of a mutant JAK3 polynucleotide or polypeptide in a biological sample (e.g. cancer) using mutant-specific reagents of the invention. Such methods may be practiced, for example, to identify a cancer, such as leukemia, that is likely to respond to an inhibitor of the kinase activity of the mutant JAK3 protein. The invention also provides, in part, methods for determining whether a compound inhibits the progression of a cancer characterized by a mutant JAK3 polypeptide. Further provided by the invention is a method for inhibiting the progression of a cancer that expresses an active mutant JAK3 polypeptide by inhibiting the expression and/or activity of the mutant polypeptide. Such methods are described in further detail below.

The further aspects, advantages, and embodiments of the invention are described in more detail below. All references cited herein are hereby incorporated by reference in their entirety.

### DEFINITIONS

As used herein, the following terms have the meanings indicated.

“Antibody” or “antibodies” refers to all types of immunoglobulins, including IgG, IgM, IgA, IgD, and IgE, including Fab or antigen-recognition fragments thereof, including chimeric, polyclonal, and monoclonal antibodies. The term “humanized antibody”, as used herein, refers to antibody molecules in which amino acids have been replaced in the non-antigen binding regions in order to more closely resemble a human antibody, while still retaining the original binding ability.

The term “biologically active” refers to a protein having structural, regulatory, or biochemical functions of a naturally occurring molecule. Likewise, “immunologically active” refers to the capability of the natural, recombinant, or synthetic mutant JAK3 polypeptide, or any oligopeptide thereof, to induce a specific immune response in appropriate animals or cells and to bind with specific antibodies.

The term “biological sample” is used in its broadest sense, and means any biological sample suspected of containing mutant JAK3 polynucleotides or polypeptides or fragments thereof, and may comprise a cell, chromosomes isolated from a cell (e.g., a spread of metaphase chromosomes), genomic DNA (in solution or bound to a solid support such as for Southern analysis), RNA (in solution or bound to a solid support such as for northern analysis), cDNA (in solution or bound to a solid support), an extract from cells, blood, urine, marrow, or a tissue, and the like. 5“Characterized by” with respect to a cancer and mutant JAK3 polynucleotide and polypeptide is meant a cancer in which a mutant JAK3 polynucleotide and/or polypeptide is/are present, as compared to a cancer in which such translocation and/or mutant polypeptide are not present. The presence of mutant polypeptide may drive, in whole or in part, the growth and survival of such cancer.

“Consensus” refers to a nucleic acid sequence which has been re-sequenced to resolve uncalled bases, or which has been extended using XL-PCR™ (Perkin Elmer, Norwalk, Conn.) in the 5′ and/or the 3′ direction and re-sequenced, or which has been assembled from the overlapping sequences of more than one Incyte clone using the GELVIEW™ Fragment Assembly system (GCG, Madison, Wis.), or which has been both extended and assembled.

“JAK3 kinase-inhibiting therapeutic” means any composition comprising one or more compounds, chemical or biological, which inhibits, either directly or indirectly, the expression and/or activity of wild type or mutant (A572V) active JAK3 kinase, either alone or as part of a mutant protein.

“Derivative” refers to the chemical modification of a nucleic acid sequence encoding mutant JAK3 polypeptide, or the encoded polypeptide itself. Illustrative of such modifications would be replacement of hydrogen by an alkyl, acyl, or amino group. A nucleic acid derivative would encode a polypeptide that retains essential biological characteristics of the natural molecule.

“Detectable label” with respect to a polypeptide, polynucleotide, or reagent disclosed herein means a chemical, biological, or other modification, including but not limited to fluorescence, mass, residue, dye, radioisotope, label, or tag modifications, etc., by which the presence of the molecule of interest may be detected.

“Expression” or “expressed” with respect to mutant JAK3 polypeptide in a biological sample means significantly expressed as compared to control sample in which this mutant polypeptide is not significantly expressed.

“Heavy-isotope labeled peptide” (used interchangeably with AQUA peptide) means a peptide comprising at least one heavy-isotope label, which is suitable for absolute quantification or detection of a protein as described in WO/03016861, “Absolute Quantification of Proteins and Modified Forms Thereof by Multistage Mass Spectrometry” (Gygi et al.), further discussed below. The term “specifically detects” with respect to such an AQUA peptide means the peptide will only detect and quantify polypeptides and proteins that contain the AQUA peptide sequence and will not substantially detect polypeptides and proteins that do not contain the AQUA peptide sequence.

“Isolated” (or “substantially purified”) refers to nucleic or amino acid sequences that are removed from their natural environment, isolated or separated. They preferably are at least 60% free, more preferably 75% free, and most preferably 90% or more free from other components with which they are naturally associated.

“Mimetic” refers to a molecule, the structure of which is developed from knowledge of the structure of mutant JAK3 polypeptide, or portions thereof and, as such, is able to effect some or all of the actions of translocation associated protein-like molecules.

“Mutant JAK3 polynucleotide” refers to the nucleic acid sequence of a substantially purified mutant JAK3 kinase (alanine 572 to valine (A572V)) gene product or polynucleotide as described herein, obtained from any species, particularly mammalian, including bovine, ovine, feline, porcine, murine, equine, and preferably human, from any source whether natural, synthetic, semi-synthetic, or recombinant.

“Mutant JAK3 polypeptide” refers to the amino acid sequence of a substantially purified mutant JAK3 kinase (alanine 572 to valine (A572V)) polypeptide described herein, obtained from any species, particularly mammalian, including bovine, ovine, feline, porcine, murine, equine, and preferably human, from any source whether natural, synthetic, semi-synthetic, or recombinant.

“Polynucleotide” (or “nucleotide sequence”) refers to an oligonucleotide, nucleotide, or polynucleotide, and fragments or portions thereof, and to DNA or RNA of genomic or synthetic origin, which may be single or double-stranded, and represent the sense or anti-sense strand.

“Polypeptide” (or “amino acid sequence”) refers to an oligopeptide, peptide, polypeptide, or protein sequence, and fragments or portions thereof, and to naturally occurring or synthetic molecules. Where “amino acid sequence” is recited herein to refer to an amino acid sequence of a naturally occurring protein molecule, “amino acid sequence” and like terms, such as “polypeptide” or “protein”, are not meant to limit the amino acid sequence to the complete, native amino acid sequence associated with the recited protein molecule.

The terms “specifically binds to” (or “specifically binding” or “specific binding”) in reference to the interaction of an antibody and a protein or peptide, mean that the interaction is dependent upon the presence of a particular structure (i.e. the antigenic determinant or epitope) on the protein; in other words, the antibody is recognizing and binding to a specific protein structure rather than to proteins in general. The term “does not bind” with respect to an antibody's binding to sequences or antigenic determinants other than that for which it is specific means does not substantially react with as compared to the antibody's binding to antigenic determinant or sequence for which the antibody is specific.

The term “stringent conditions” with respect to sequence or probe hybridization conditions is the “stringency” that occurs within a range from about Tₘ minus 5° C. (5° C. below the melting temperature (Tₘ) of the probe or sequence) to about 20° C. to 25° C. below Tₘ. Typical stringent conditions are: overnight incubation at 42° C. in a solution comprising: 50% formamide, 5×.SSC (750 mM NaCl, 75 mM trisodium citrate), 50 mM sodium phosphate (pH 7.6), 5×Denhardt's solution, 10% dextran sulfate, and 20 micrograms/ml denatured, sheared salmon sperm DNA, followed by washing the filters in 0.1×SSC at about 65° C. As will be understood by those of skill in the art, the stringency of hybridization may be altered in order to identify or detect identical or related polynucleotide sequences.

A “variant” of a mutant JAK3 polypeptide refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions, or both. Guidance in determining which amino acid residues may be substituted, inserted, or deleted without abolishing biological or immunological activity may be found using computer programs well known in the art, for example, DNASTAR software.

### A. Identification of Mutant JAK3 Kinase in Human Leukemia.

The novel JAK3 kinase activating mutation disclosed herein, which occurs at residue 572 (alanine to valine), was surprisingly identified during examination of global phosphorylated peptide profiles in extracts from a cell line (CMK) of human acute megakaryoblastic leukemia (AML-M7), a subtype of acute myelogenous leukemia (AML).

The phosphorylation profile of this cell line was elucidated using a recently described technique for the isolation and mass spectrometric characterization of modified peptides from complex mixtures (see U.S. Patent Publication No. 20030044848, Rush et al., “Immunoaffinity Isolation of Modified Peptides from Complex Mixtures” (the “IAP” technique), as further described in Example 2 below.

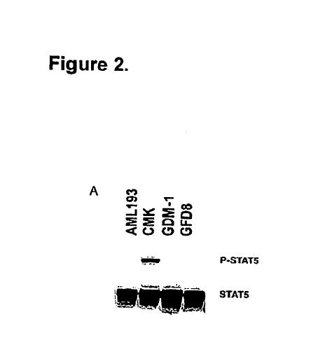

STAT5 has been found to be constitutively tyrosine phosphorylated in the leukemic cells of approximately 70% of patients with acute myeloid leukemia (AML). See, e.g. Birkenkamp et al., Leukemia 15:1923-1931 (2001). The presence of FLT3 or KIT activating mutations can account for STAT5 phosphorylation in up to 35% of patients. However, constitutive STAT5 phosphorylation is detected in a significant percentage of patients lacking these mutations. Accordingly, immunoblot analysis using a phospho-STAT5 antibody was used to screen several human AML cell lines for constitutive phosphorylation of STAT5, as further described in Example 1. STAT5 was found to be constitutively activated in CMK cells (see FIG. 2A), a megakaryoblastic cell line derived from a patient with Down syndrome and M7 leukemia. Activating FLT3 or KIT mutations are commonly responsible for constitutive activation of STAT5 in AML, however, denaturing-HPLC analysis revealed that the CMK cell line does not possess activating FLT3 or KIT mutations (see Example 1).

Application of the IAP technique using a phosphotyrosine-specific antibody (CELL SIGNALING TECHNOLOGY, INC., Beverly, Mass., 2003/04 Cat. #9411), in order to identify the upstream activator of STAT5, identified that the CMK cell line expresses JAK3 kinase (see FIG. 2B). As shown in FIG. 2B, several kinases were identified as being tyrosine phosphorylated and/or activated in this cell line, including JAK3 and TYK2.

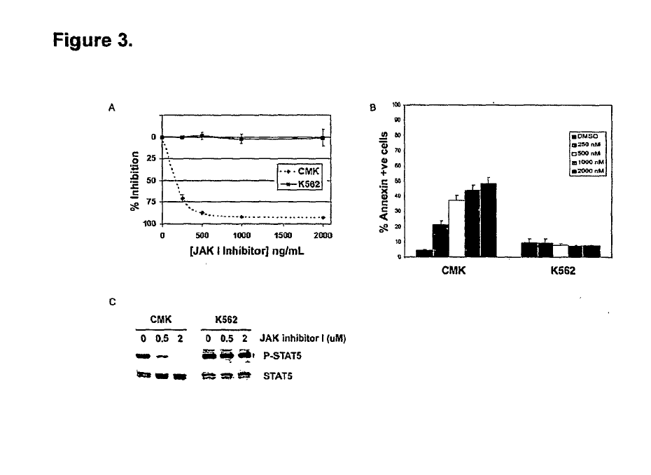

To confirm that JAK3, which is known to directly phosphorylate STAT proteins, was activated and driving the growth and/or viability of the CMK cell line, a pan-JAK kinase inhibitor (JAK I) was employed. As expected, the inhibitor had no effect on the control cell line (K562), while cell proliferation and viability, as well STAT5 phosphorylation, were significantly decreased in the CMK cell line. See Example 3 and FIG. 3.

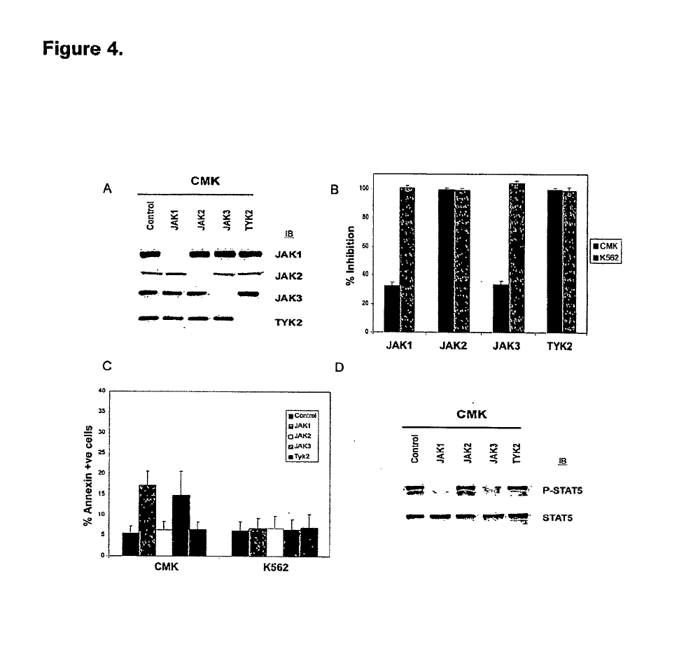

siRNA induced down-regulation of JAK family kinases was employed to further confirm that activated JAK3 is driving cell proliferation, survival, and STAT5 phosphorylation, in the CMK AML cell line. See Example 4 and FIG. 4. Interestingly, JAK1 kinase inhibition was also found to effect growth and survival of the CMK cell line, and simultaneous inhibition of both JAK3 and JAK1 resulted in a cooperative effect.

Mutational analysis was then employed to determine whether any mutations existed in the kinase or JH2 pseudokinase domains of JAK3 and JAK1 expressed by the CMK cell line. This analysis revealed the presence of a novel point mutation (A572V) in the JH2 domain of JAK3. See Example 5. siRNA down regulation of JAK3 and JAK1 was again employed to establish that the mutant (A572V) JAK3 kinase interacts with (co-precipitates with) JAK1 and is responsible for activating JAK1, which in turn directly phosphorylates STAT5 in the AML cell line (CMK). See Example 6.

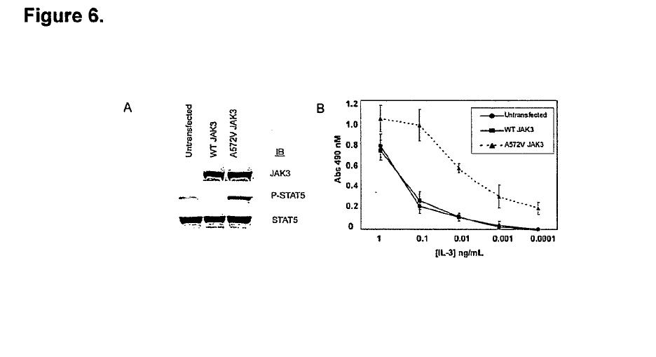

cDNA encoding the mutant JAK 3 kinase was prepared by site-directed mutagenesis, and transfected into a murine hematopoietic progenitor cell line (BaF3) to confirm that expression of the mutant (A572V) JAK3 kinase transforms the cells and drives proliferation and growth of these cells (see Example 7 and FIG. 6).

### B. Isolated Polynucleotides.

The present invention provides, in part, isolated polynucleotides that encode mutant (A572V) JAK3 polypeptides, nucleotide probes that hybridize to such polynucleotides, and methods, vectors, and host cells for utilizing such polynucleotides to produce recombinant mutant polypeptides.

Unless otherwise indicated, all nucleotide sequences determined by sequencing a DNA molecule herein were determined using an automated DNA sequencer (such as the Model 373 from Applied Biosystems, Inc.), and all amino acid sequences of polypeptides encoded by DNA molecules determined herein were determined using an automated peptide sequencer. As is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Unless otherwise indicated, each nucleotide sequence set forth herein is presented as a sequence of deoxyribonucleotides (abbreviated A, G, C and T). However, by “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U). For instance, reference to an RNA molecule having the sequence of SEQ ID NO: 2 set forth using deoxyribonucleotide abbreviations is intended to indicate an RNA molecule having a sequence in which each deoxyribonucleotide A, G or C of SEQ ID NO: 2 has been replaced by the corresponding ribonucleotide A, G or C, and each deoxyribonucleotide T has been replaced by a ribonucleotide U.

In one embodiment, the invention provides an isolated polynucleotide comprising a nucleotide sequence at least 95% identical to a sequence selected from the group consisting of:

(a) a nucleotide sequence encoding a mutant Janus Kinase-3 (JAK3) polypeptide comprising the amino acid sequence of SEQ ID NO: 1 but having an alanine-to-valine point mutation at residue 572;

(b) a nucleotide sequence encoding a mutant JAK3 kinase polypeptide, said nucleotide sequence comprising the nucleotide sequence of SEQ ID NO: 2 but having an alanine-to-valine codon mutation at nucleotides 1773-1775;

(c) a nucleotide sequence encoding a mutant JAK3 kinase polypeptide comprising the kinase domain (residues 822-1095 of SEQ ID NO: 1) and JH2-pseudokinase domain (residues 521-777 of SEQ ID NO: 1) of JAK3 kinase, but having an alanine-to-valine point mutation at residue 572 in the JH2-pseudokinase domain;

(d) a nucleotide sequence encoding a mutant JAK3 kinase polypeptide, said nucleotide sequence comprising the nucleotide sequences encoding the kinase domain of JAK3 (nucleotides 822-1095 of SEQ ID NO: 2) and the JH2-pseudokinase domain of JAK3 (nucleotides 521-777 of JAK3), but having an alanine-to-valine codon mutation at nucleotides 1773-1775 in the JH2-pseudokinase domain;

(e) a mutant JAK3 kinase nucleotide sequence comprising at least six contiguous nucleotides encompassing an alanine-to-valine codon mutation at nucleotides 1773-1775 of SEQ ID NO: 2;

(f) a nucleotide sequence encoding a mutant JAK3 polypeptide comprising at least six contiguous amino acids encompassing an alanine-to-valine point mutation at residue 572 of SEQ ID NO: 1; and

(g) a nucleotide sequence complementary to any of the nucleotide sequences of (a)-(f).

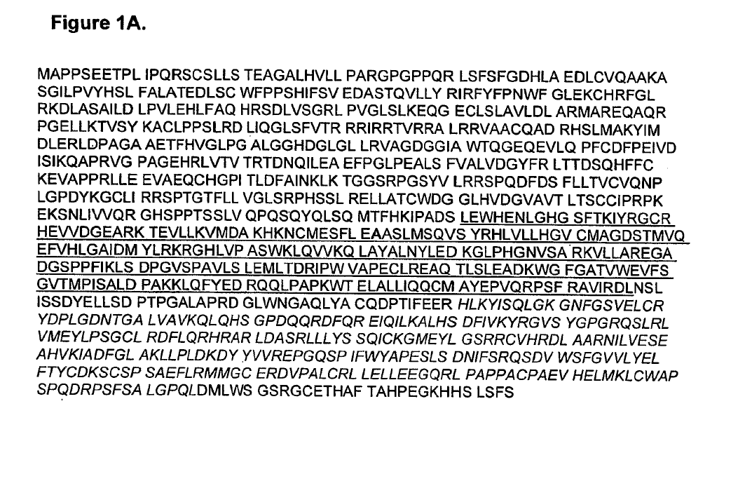

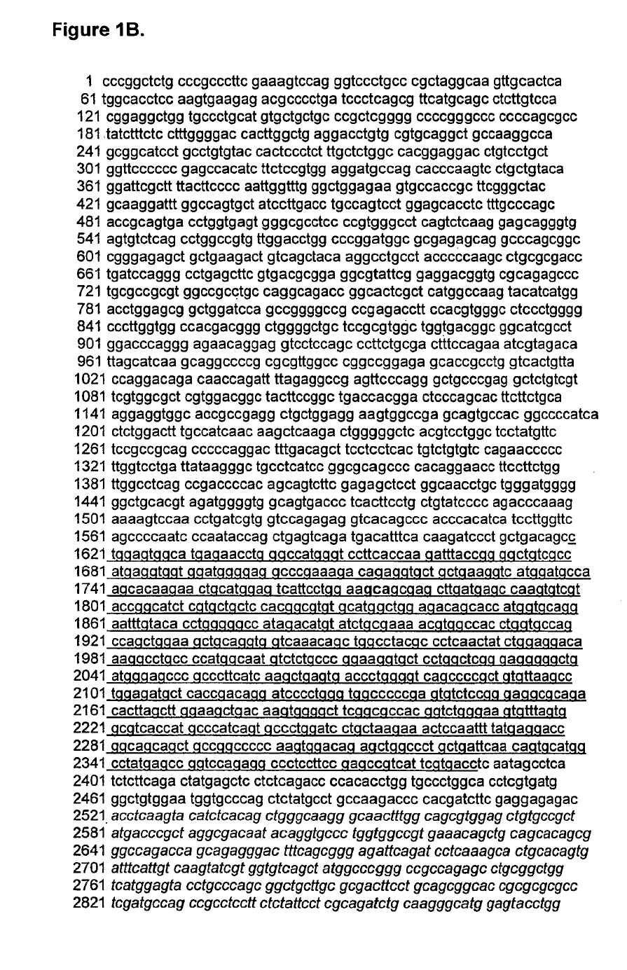

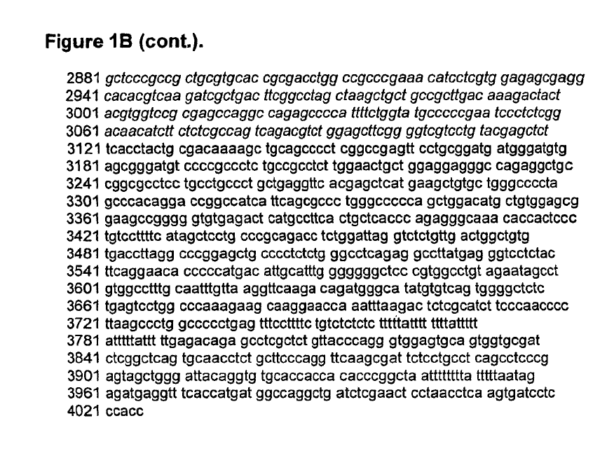

Using the information provided herein, such as the nucleotide sequence in FIG. 1B (SEQ ID NO: 2), a nucleic acid molecule of the present invention encoding a mutant JAK3 polypeptide of the invention may be obtained using standard cloning and screening procedures, such as those for cloning cDNAs using mRNA as starting material. Illustrative of the invention, the mutant JAK3 polynucleotide described in FIG. 1B (SEQ ID NO: 2) (but having the alanine-to-valine codon mutation at nucleotides 1773-1775), identified in a human AML cell line, was cloned in a cDNA and used to transform a murine cell line (as further described in Example 7 below). The mutant (A572V) JAK3 kinase gene can also be identified in genomic DNA or cDNA libraries in other leukemias or cancers in which this point mutation occurs.

The nucleotide sequence of the mutant JAK3 kinase gene (SEQ ID NO: 2—but having an alanine-to-valine codon mutation at nucleotides 1773-1775) encodes a constitutively active mutant JAK3 kinase protein of 1124 amino acid residues (see FIG. 1A (SEQ ID NO: 1) containing the A572V point mutation. The full-length mutant JAK3 polynucleotide comprises the portions of the nucleotide sequence of wild type JAK3 that encode the kinase domain (amino acids 822-1095) and JH2-pseudokinase domain (amino acids 521-777), but having an alanine-to-valine codon mutation for amino acid 572. One of skill in the art can readily determine if a codon for alanine (GCU, GCC, GCA, GCG) is mutated to a codon for valine (GUU, GUC, GUA, GUG).

As indicated, the present invention provides, in part, the mature form of the mutant (A572) JAK3 kinase, which remains functional and is constitutively active. According to the signal hypothesis, proteins secreted by mammalian cells have a signal or secretory leader sequence which is cleaved from the mature protein once export of the growing protein chain across the rough endoplasmic reticulum has been initiated. Most mammalian cells and even insect cells cleave secreted proteins with the same specificity. However, in some cases, cleavage of a secreted protein is not entirely uniform, which results in two or more mature species on the protein. Further, it has long been known that the cleavage specificity of a secreted protein is ultimately determined by the primary structure of the complete protein, that is, it is inherent in the amino acid sequence of the polypeptide. Therefore, the present invention provides, in part, nucleotide sequences encoding a mature mutant JAK3 kinase polypeptide having the amino acid sequence encoded by the cDNA clone identified as ATCC Deposit No. ______, which was deposited with the American Type Culture Collection (Manassas, Va., U.S.A.) on Jan. 18, 2007 in accordance with the provisions of the Budapest Treaty.

By the mature mutant JAK3 polypeptide having the amino acid sequence encoded by the deposited cDNA clone is meant the mature form of this mutant protein produced by expression in a mammalian cell (e.g., COS cells, as described below) of the complete open reading frame encoded by the human DNA sequence of the deposited clone.

As indicated, polynucleotides of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

Isolated polynucleotides of the invention are nucleic acid molecules, DNA or RNA, which have been removed from their native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated polynucleotides of the invention include the DNA molecule shown in FIG. 1B (SEQ ID NO: 2)—but having an alanine-to-valine codon mutation for amino acid 572, DNA molecules comprising the coding sequence for the mature mutant JAK3 protein shown in FIG. 1A (SEQ ID NO: 1), and DNA molecules that comprise a sequence substantially different from those described above but which, due to the degeneracy of the genetic code, still encode a JAK3 mutant polypeptide of the invention. The genetic code is well known in the art, thus, it would be routine for one skilled in the art to generate such degenerate variants.

In another embodiment, the invention provides an isolated polynucleotide encoding a mutant JAK3 kinase polypeptide comprising the mutant (A572V), JAK3 kinase nucleotide sequence contained in the above-described deposited cDNA clone. Preferably, such nucleic acid molecule will encode the mature mutant polypeptide encoded by the deposited cDNA clone. In another embodiment, the invention provides an isolated nucleotide sequence encoding a mutant JAK3 kinase polypeptide comprising the kinase domain (residues 822-1095 of SEQ ID NO: 1) and JH2-pseudokinase domain (residues 521-777 of SEQ ID NO: 1) of JAK3 kinase, but having an alanine-to-valine point mutation at residue 572 in the JH2-pseudokinase domain. In another embodiment, there is provided an isolated nucleotide sequence encoding a mutant JAK3 kinase polypeptide, said nucleotide sequence comprising the nucleotide sequences encoding the kinase domain of JAK3 (nucleotides 2523-3342 of SEQ ID NO: 2) and the JH2-pseudokinase domain of JAK3 (nucleotides 1620-2388 of JAK3), but having an alanine-to-valine codon mutation at nucleotides 1773-1775 in the JH2-pseudokinase domain;

The invention further provides isolated polynucleotides comprising nucleotide sequences having a sequence complementary to one of the mutant JAK3 polynucleotides of the invention. Such isolated molecules, particularly DNA molecules, are useful as probes for gene mapping, by in situ hybridization with chromosomes, and for detecting expression of the mutant JAK3 kinase protein in human tissue, for instance, by Northern blot analysis, as further described in Section F below.

The present invention is further directed to fragments of the isolated nucleic acid molecules described herein. By a fragment of an isolated mutant JAK3 polynucleotide of the invention is intended fragments at least about 15 nucleotides, and more preferably at least about 20 nucleotides, still more preferably at least about 30 nucleotides, and even more preferably, at least about 40 nucleotides in length, which are useful as diagnostic probes and primers as discussed herein. Of course, larger fragments of about 50-1500 nucleotides in length are also useful according to the present invention, as are fragments corresponding to most, if not all, of the mutant JAK3 nucleotide sequence of the deposited cDNA or as shown in FIG. 1B (SEQ ID NO: 2). By a fragment at least 20 nucleotides in length, for example, is intended fragments which include 20 or more contiguous bases from the respective nucleotide sequences from which the fragments are derived.

Generation of such DNA fragments is routine to the skilled artisan, and may be accomplished, by way of example, by restriction endonuclease cleavage or shearing by sonication of DNA obtainable from the deposited cDNA clone or synthesized according to the sequence disclosed herein. Alternatively, such fragments can be directly generated synthetically.

Preferred nucleic acid fragments or probes of the present invention include nucleic acid molecules encoding and encompassing the mutation point (amino acid 572) in wild type JAK3 (see FIGS. 1A-1B. For example, in certain preferred embodiments, an isolated mutant JAK3 polynucleotide of the invention comprises a JAK3 kinase nucleotide sequence/fragment comprising at least six contiguous nucleotides encompassing an alanine-to-valine codon mutation at nucleotides 1773-1775 of SEQ ID NO: 2. In another preferred embodiment, an isolated mutant JAK3 polynucleotide of the invention comprises a nucleotide sequence/fragment encoding a mutant JAK3 polypeptide comprising at least six contiguous amino acids encompassing an alanine-to-valine point mutation at residue 572 of SEQ ID NO: 1.

In another aspect, the invention provides an isolated polynucleotide that hybridizes under stringent hybridization conditions to a portion of a mutant JAK3 polynucleotide of the invention as described herein. By “stringent hybridization conditions” is intended overnight incubation at 42° C. in a solution comprising: 50% formamide, 5×.SSC (750 mM NaCl, 75 mM trisodium citrate), 50 mM sodium phosphate (pH 7.6), 5×Denhardt's solution, 10% dextran sulfate, and 20 micrograms/ml denatured, sheared salmon sperm DNA, followed by washing the filters in 0.1×SSC at about 65° C.

By a polynucleotide that hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 nt of the reference polynucleotide. These are useful as diagnostic probes and primers as discussed above and in more detail below.

Of course, polynucleotides hybridizing to a larger portion of the reference polynucleotide (e.g. the mature mutant JAK3 kinase polynucleotide described in FIG. 1B (SEQ ID NO: 2)—but having an alanine-to-valine codon mutation for amino acid 572), for instance, a portion 50-750 nt in length, or even to the entire length of the reference polynucleotide, are also useful as probes according to the present invention, as are polynucleotides corresponding to most, if not all, of the nucleotide sequences of the deposited cDNA or the nucleotide sequences shown in FIG. 1B (SEQ ID NO: 2).

By a portion of a polynucleotide of “at least 20 nucleotides in length,” for example, is intended 20 or more contiguous nucleotides from the nucleotide sequence of the reference polynucleotide. As indicated, such portions are useful diagnostically either as a probe according to conventional DNA hybridization techniques or as primers for amplification of a target sequence by the polymerase chain reaction (PCR), as described, for instance, in MOLECULAR CLONING, A LABORATORY MANUAL, 2nd. edition, Sambrook, J., Fritsch, E. F. and Maniatis, T., eds., Cold Spring Harbor Laboratory Press, Cold Spring Harbor, N.Y. (1989), the entire disclosure of which is hereby incorporated herein by reference. Of course, a polynucleotide which hybridizes only to a poly A sequence (such as the 3′ terminal poly(A) tract of the JAK3 kinase sequence shown in FIG. 1B (SEQ ID NO: 2)) or to a complementary stretch of T (or U) resides, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention, which encode a mutant JAK3 polypeptide of the invention, may include but are not limited to those encoding the amino acid sequence of the mature polypeptide, by itself; the coding sequence for the mature polypeptide and additional sequences, such as those encoding the leader or secretory sequence, such as a pre-, or pro- or pre-pro-protein sequence; the coding sequence of the mature polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities.

Thus, the sequence encoding the polypeptide may be fused to a marker sequence, such as a sequence encoding a peptide that facilitates purification of the fused polypeptide. In certain preferred embodiments of this aspect of the invention, the marker amino acid sequence is a hexa-histidine peptide, such as the tag provided in a pQE vector (Qiagen, Inc.), among others, many of which are commercially available. As described in Gentz et al., Proc. Natl. Acad. Sci. USA 86: 821-824 (1989), for instance, hexa-histidine provides for convenient purification of the mutant protein. The “HA” tag is another peptide useful for purification which corresponds to an epitope derived from the influenza hemagglutinin protein, which has been described by Wilson et al., Cell 37: 767 (1984). As discussed below, other such mutant proteins include the mutant JAK3 kinase polypeptide itself fused to F_{c} at the N- or C-terminus.

The present invention further relates to variants of the nucleic acid molecules of the present invention, which encode portions, analogs or derivatives of a mutant JAK3 kinase polypeptide disclosed herein. Variants may occur naturally, such as a natural allelic variant. By an “allelic variant” is intended one of several alternate forms of a gene occupying a given locus on a chromosome of an organism. See, e.g. GENES II, Lewin, B., ed., John Wiley & Sons, New York (1985) Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities (e.g. kinase activity) of the mutant JAK3 polypeptides disclosed herein. Also especially preferred in this regard are conservative substitutions.

Further embodiments of the invention include isolated polynucleotides comprising a nucleotide sequence at least 90% identical, and more preferably at least 95%, 96%, 97%, 98% or 99% identical, to a mutant JAK3 polynucleotide of the invention (for example, a nucleotide sequence encoding the mutant JAK3 kinase polypeptide having the complete amino acid sequence shown in FIG. 1A (SEQ ID NO: 1)—but having the A572V codon mutation; or a nucleotide sequence encoding the kinase and JH2-pseudokinase domains of JAK3 (see FIG. 1A) and having the A572V codon mutation within the JH2 domain); or a nucleotide complementary to such exemplary sequences).

By a polynucleotide having a nucleotide sequence at least, for example, 95% “identical” to a reference nucleotide sequence encoding a mutant JAK3 polypeptide is intended that the nucleotide sequence of the polynucleotide is identical to the reference sequence except that the polynucleotide sequence may include up to five point mutations per each 100 nucleotides of the reference nucleotide sequence encoding the mutant JAK3 polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequence shown in FIG. 1B (SEQ ID NO: 2) or to the nucleotide sequence of the deposited cDNA clone described above can be determined conventionally using known computer programs such as the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference mutant JAK3 kinase polynucleotide sequence according to the present invention, the parameters are set, of course, such that the percentage of identity is calculated over the full length of the reference nucleotide sequence and that gaps in homology of up to 5% of the total number of nucleotides in the reference sequence are allowed.

The present invention includes in its scope nucleic acid molecules at least 90%, 95%, 96%, 97%, 98% or 99% identical to the nucleic acid sequence shown in FIG. 1B (SEQ ID NO: 2) which comprise an alanine-to-valine codon mutation for amino acid 572, irrespective of whether they encode a polypeptide having JAK3 kinase activity. This is because even where a particular mutant nucleic acid molecule does not encode a mutant polypeptide having JAK3 kinase activity, one of skill in the art would still know how to use the nucleic acid molecule, for instance, as a hybridization probe or a polymerase chain reaction (PCR) primer. Uses of the nucleic acid molecules of the present invention that do not encode a mutant JAK3 polypeptide having kinase include, inter alia, (1) isolating the mutant JAK3 kinase gene, or allelic variants thereof in a cDNA library; (2) in situ hybridization (e.g., “FISH”) to metaphase chromosomal spreads to provide precise chromosomal location of the mutated JAK3 gene, as described in Verma et al., HUMAN CHROMOSOMES: A MANUAL OF BASIC TECHNIQUES, Pergamon Press, New York (1988); and Northern Blot analysis for detecting mutant JAK3 kinase protein or mRNA expression in specific tissues.

Preferred, however, are nucleic acid molecules having sequences at least 95% identical to a mutant JAK3 polypeptide of the invention or to the nucleic acid sequence of the deposited cDNA, which do, in fact, encode a mutant polypeptide having JAK3 kinase activity (as disclosed in the Examples). Such activity may be similar, but not necessarily identical, to the constitutive activity of the mutant JAK3 kinase protein disclosed herein (either the full-length protein, the mature protein, or a protein fragment that retains kinase activity), as measured in a particular biological assay. For example, the kinase activity of JAK3 can be examined by determining its ability to phosphorylate and activate the STAT family of proteins, which are substrates for the JAK family of kinases.

Due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNA or the nucleic acid sequence shown in FIG. 1B (SEQ ID NO: 2)—but having an alanine-to-valine codon mutation for amino acid 572—will encode a mutant polypeptide having JAK3 activity. In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide that retains JAK3 kinase activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie et al., “Deciphering the Message in Protein Sequences: Tolerance to Amino Acid Substitutions,” Science 247:1306-1310 (1990), which describes two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions. Skilled artisans familiar with such techniques also appreciate which amino acid changes are likely to be permissive at a certain position of the protein. For example, most buried amino acid residues require nonpolar side chains, whereas few features of surface side chains are generally conserved. Other such phenotypically silent substitutions are described in Bowie et al., supra., and the references cited therein.

Methods for DNA sequencing that are well known and generally available in the art may be used to practice any polynucleotide embodiments of the invention. The methods may employ such enzymes as the Klenow fragment of DNA polymerase I, SEQUENASE® (US Biochemical Corp, Cleveland, Ohio), Taq polymerase (Perkin Elmer), thermostable T7 polymerase (Amersham, Chicago, Ill.), or combinations of recombinant polymerases and proofreading exonucleases such as the ELONGASE Amplification System marketed by Gibco BRL (Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI 377 DNA sequencers (Perkin Elmer).

Polynucleotide sequences encoding a mutant JAK3 polypeptide of the invention may be extended utilizing a partial nucleotide sequence and employing various methods known in the art to detect upstream sequences such as promoters and regulatory elements. For example, one method that may be employed, “restriction-site” PCR, uses universal primers to retrieve unknown sequence adjacent to a known locus (Sarkar, G., PCR Methods Applic. 2:318-322 (1993)). In particular, genomic DNA is first amplified in the presence of primer to linker sequence and a primer specific to the known region. Exemplary primers are those described in Example 7 herein. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region (Triglia et al., Nucleic Acids Res. 16: 8186 (1988)). The primers may be designed using OLIGO 4.06 Primer Analysis software (National Biosciences Inc., Plymouth, Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome DNA (Lagerstrom et al., PCR Methods Applic. 1: 111-119 (1991)). In this method, multiple restriction enzyme digestions and ligations may also be used to place an engineered double-stranded sequence into an unknown portion of the DNA molecule before performing PCR. Another method which may be used to retrieve unknown sequences is that described in Parker et al., Nucleic Acids Res. 19: 3055-3060 (1991)). Additionally, one may use PCR, nested primers, and PROMOTERFINDER® libraries to walk in genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into the 5′ and 3′ non-transcribed regulatory regions.

Capillary electrophoresis systems, which are commercially available, may be used to analyze the size or confirm the nucleotide sequence of sequencing or PCR products. In particular, capillary sequencing may employ flowable polymers for electrophoretic separation, four different fluorescent dyes (one for each nucleotide), which are laser activated, and detection of the emitted wavelengths by a charge coupled device camera. Output/light intensity may be converted to electrical signal using appropriate software (e.g. GENOTYPER™ and SEQUENCE NAVIGATOR™, Perkin Elmer) and the entire process from loading of samples to computer analysis and electronic data display may be computer controlled. Capillary electrophoresis is especially preferable for the sequencing of small pieces of DNA that might be present in limited amounts in a particular sample.

### C. Vectors and Host Cells.

The present invention also provides recombinant vectors that comprise an isolated mutant JAK3 polynucleotide of the present invention, host cells which are genetically engineered with the recombinant vectors, and the production of recombinant mutant JAK3 kinase polypeptides or fragments thereof by recombinant techniques.

Recombinant constructs may be introduced into host cells using well-known techniques such infection, transduction, transfection, transvection, electroporation and transformation. The vector may be, for example, a phage, plasmid, viral or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

Preferred vectors comprise cis-acting control regions to the polynucleotide of interest. Appropriate trans-acting factors may be supplied by the host, supplied by a complementing vector or supplied by the vector itself upon introduction into the host. In certain preferred embodiments in this regard, the vectors provide for specific expression, which may be inducible and/or cell type-specific. Particularly preferred among such vectors are those inducible by environmental factors that are easy to manipulate, such as temperature and nutrient additives.

Expression vectors useful in the present invention include chromosomal-, episomal- and virus-derived vectors, e.g., vectors derived from bacterial plasmids, bacteriophage, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids.

The DNA insert comprising a mutant (A572V) JAK3 polynucleotide of the invention should be operatively linked to an appropriate promoter, such as the phage lambda PL promoter, the E. coli lac, trp and tac promoters, the SV40 early and late promoters and promoters of retroviral LTRs, to name a few. Other suitable promoters are known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Among known bacterial promoters suitable for use in the present invention include the E. Coli lad and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-1 promoter.

In the yeast, Saccharomyces cerevisiae, a number of vectors containing constitutive or inducible promoters such as alpha factor, alcohol oxidase, and PGH may be used. For reviews, see Ausubel et al. (1989) CURRENT PROTOCOLS IN MOLECULAR BIOLOGY, John Wiley & Sons, New York, N.Y., and Grant et al., Methods Enzymol. 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., BASIC METHODS IN MOLECULAR BIOLOGY (1986).

Transcription of DNA encoding a mutant JAK3 kinase polypeptide of the present invention by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at basepairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide may be expressed in a modified form, such as a mutant protein (e.g. a GST-mutant), and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred mutant protein comprises a heterologous region from immunoglobulin that is useful to solubilize proteins.

Mutant JAK3 polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Accordingly, in one embodiment, the invention provides a method for producing a recombinant mutant JAK3 kinase polypeptide by culturing a recombinant host cell (as described above) under conditions suitable for the expression of the mutant polypeptide and recovering the polypeptide. Culture conditions suitable for the growth of host cells and the expression of recombinant polypeptides from such cells are well known to those of skill in the art. See, e.g., CURRENT PROTOCOLS IN MOLECULAR BIOLOGY, Ausubel F M et al., eds., Volume 2, Chapter 16) Wiley Interscience.

### D. Isolated Polypeptides.

The invention also provides, in part, isolated mutant JAK3 kinase polypeptides and fragments thereof. In one embodiment, the invention provides an isolated polypeptide comprising an amino acid sequence at least 95% identical to a sequence selected from the group consisting of (a) an amino acid sequence encoding a mutant Janus Kinase-3 (JAK3) polypeptide comprising the amino acid sequence of SEQ ID NO: 1 but having an alanine-to-valine point mutation at residue 572;

(b) an amino acid sequence encoding a mutant JAK3 kinase polypeptide comprising the kinase domain (residues 822-1095 of SEQ ID NO: 1) and the JH2 pseudokinase domain (residues 521-777 of SEQ ID NO: 1), but having an alanine-to-valine point mutation at residue 572 in the JH2 pseudo-kinase domain; and

(c) an amino acid sequence encoding a mutant JAK3 polypeptide comprising at least six contiguous amino acids encompassing residue 572 of SEQ ID NO: 1, but having an alanine-to-valine point mutation at this position.

As disclosed herein, full-length mutant (A572V) JAK3 kinase polypeptide not only retains kinase activity, but the mutation results in a constitutively active kinase.

In one preferred embodiment, the invention provides an isolated mutant JAK3 kinase polypeptide having the amino acid sequence encoded by the deposited cDNA described above (ATCC Deposit No. ______). In another preferred embodiment, recombinant mutant JAK3 polypeptides of the invention are provided, which may be produced using a recombinant vector or recombinant host cell as described above.

It will be recognized in the art that some amino acid sequences of a mutant JAK3 kinase polypeptide can be varied without significant effect of the structure or function of the mutant protein. If such differences in sequence are contemplated, it should be remembered that there will be critical areas on the protein which determine activity (e.g. the kinase and JH2 domains of JAK3). In general, it is possible to replace residues that form the tertiary structure, provided that residues performing a similar function are used. In other instances, the type of residue may be completely unimportant if the alteration occurs at a non-critical region of the protein.

Thus, the invention further includes variations of a mutant JAK3 kinase polypeptide that retain substantial JAK3 kinase activity. Such mutants include deletions, insertions, inversions, repeats, and type substitutions (for example, substituting one hydrophilic residue for another, but not strongly hydrophilic for strongly hydrophobic as a rule). Small changes or such “neutral” amino acid substitutions will generally have little effect on activity.

Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Examples of conservative amino acid substitutions known to those skilled in the art are: Aromatic: phenylalanine tryptophan tyrosine; Hydrophobic: leucine isoleucine valine; Polar: glutamine asparagines; Basic: arginine lysine histidine; Acidic: aspartic acid glutamic acid; Small: alanine serine threonine methionine glycine. As indicated in detail above, further guidance concerning which amino acid changes are likely to be phenotypically silent (i.e., are not likely to have a significant deleterious effect on a function) can be found in Bowie et al., Science 247, supra.

The polypeptides of the present invention are preferably provided in an isolated form, and preferably are substantially purified. A recombinantly produced version of a mutant JAK3 kinase polypeptide of the invention can be substantially purified by the one-step method described in Smith and Johnson, Gene 67: 31-40 (1988).

The polypeptides of the present invention include the mutant JAK3 kinase polypeptide of FIG. 1A (SEQ ID NO: 1)—but wherein alanine 572 is mutated to valine—(whether or not including a leader sequence), the mutant polypeptide encoded by the deposited cDNA clone (ATCC No.) ______), an amino acid sequence encoding a mutant JAK3 kinase polypeptide comprising the kinase and JH2 pseudo-kinase domains of JAK3 (residues 822-1095 and 521-777 of SEQ ID NO: 1, respectively), but having the A572V mutation, and an amino acid sequence encoding a polypeptide comprising at least six contiguous amino acids encompassing alanine 572 of SEQ ID NO: 1, but wherein this position is mutated to valine, as well as polypeptides that have at least 90% similarity, preferably at least 95% similarity, and still more preferably at least 96%, 97%, 98% or 99% similarity to those described above and comprising the A572V mutation.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2: 482-489 (1981)) to find the best segment of similarity between two sequences.

By a polypeptide having an amino acid sequence at least, for example, 95% “identical” to a reference amino acid sequence of a mutant JAK3 kinase polypeptide of the invention is intended that the amino acid sequence of the polypeptide is identical to the reference sequence except that the polypeptide sequence may include up to five amino acid alterations per each 100 amino acids of the reference amino acid sequence of the mutant JAK3 polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

A mutant JAK3 kinase polypeptide of the present invention may be purified using a molecular weight marker on SDS-PAGE gels or on molecular sieve gel filtration columns, for example, using methods well known to those of skill in the art.

As further described in detail below, the polypeptides of the present invention can also be used to generate mutant polypeptide-specific reagents, such as polyclonal and monoclonal antibodies, which are useful in assays for detecting mutant (A572V) JAK3 polypeptide expression as described below, or as agonists and antagonists capable of enhancing or inhibiting the function/activity of the mutant JAK3 protein. Further, such polypeptides can be used in the yeast two-hybrid system to “capture” mutant JAK3 kinase polypeptide binding proteins, which are also candidate agonist and antagonist according to the present invention. The yeast two hybrid system is described in Fields and Song, Nature 340: 245-246 (1989).

In another aspect, the invention provides a peptide or polypeptide comprising an epitope-bearing portion of a polypeptide of the invention, for example, an epitope comprising the mutation site (A572) of a mutant JAK3 kinase polypeptide. The epitope of this polypeptide portion is an immunogenic or antigenic epitope of a polypeptide of the invention. An “immunogenic epitope” is defined as a part of a protein that elicits an antibody response when the whole protein is the immunogen. These immunogenic epitopes are believed to be confined to a few loci on the molecule. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983). The production of mutant JAK3 kinase polypeptide-specific antibodies of the invention is described in further detail below.

The antibodies raised by antigenic epitope-bearing peptides or polypeptides are useful to detect a mimicked protein, and antibodies to different peptides may be used for tracking the fate of various regions of a protein precursor which undergoes post-translational processing. The peptides and anti-peptide antibodies may be used in a variety of qualitative or quantitative assays for the mimicked protein, for instance in competition assays since it has been shown that even short peptides (e.g., about 9 amino acids) can bind and displace the larger peptides in immunoprecipitation assays. See, e.g., Wilson et al., Cell 37: 767-778 (1984) at 777. The anti-peptide antibodies of the invention also are useful for purification of the mimicked protein, for instance, by adsorption chromatography using methods well known in the art. Immunological assay formats are described in further detail below.

Recombinant mutant JAK3 polypeptides are also within the scope of the present invention, and may be producing using polynucleotides of the invention, as described in Section B above. For example, the invention provides, in part, a method for producing a recombinant mutant JAK3 kinase polypeptide by culturing a recombinant host cell (as described above) under conditions suitable for the expression of the mutant polypeptide and recovering the mutant polypeptide. Culture conditions suitable for the growth of host cells and the expression of recombinant polypeptides from such cells are well known to those of skill in the art.

### E. Mutant-Specific Reagents

Mutant JAK3 polypeptide-specific reagents useful in the practice of the disclosed methods include, among others, mutant polypeptide specific antibodies and AQUA peptides (heavy-isotope labeled peptides) corresponding to, and suitable for detection and quantification of, mutant (A572V) JAK3 kinase polypeptide expression in a biological sample. A mutant polypeptide-specific reagent is any reagent, biological or chemical, capable of specifically binding to, detecting and/or quantifying the presence/level of expressed mutant JAK3 kinase polypeptide of the invention in a biological sample, while not binding to or detecting wild type JAK3. The term includes, but is not limited to, the preferred antibody and AQUA peptide reagents discussed below, and equivalent reagents are within the scope of the present invention.

### Antibodies.

Reagents suitable for use in practice of the methods of the invention include a mutant JAK3 kinase polypeptide-specific antibody. A mutant-specific antibody of the invention is an isolated antibody or antibodies that specifically bind(s) a mutant (A572V) JAK3 kinase polypeptide of the invention, but does not substantially bind either wild type JAK3 or JAK3 kinase when mutated at other positions.

Mutant polypeptide-specific antibodies generated against human mutant JAK3 may also bind to highly homologous and equivalent epitopic peptide sequences in other mammalian species, for example murine, rat, feline, pig, or rabbit, and vice versa. Antibodies useful in practicing the methods of the invention include (a) monoclonal antibodies, (b) purified polyclonal antibodies that specifically bind to the target polypeptide (e.g. an epitope comprising the A572 mutation point of JAK3 kinase, (c) antibodies as described in (a)-(b) above that bind equivalent and highly homologous epitopes or phosphorylation sites in other non-human species (e.g. mouse, rat), and (d) fragments of (a)-(c) above that bind to the antigen (or more preferably the epitope) bound by the exemplary antibodies disclosed herein.

The term “antibody” or “antibodies” as used herein refers to all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81:6851 (1984); Neuberger et al., Nature 312:604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed specific antibodies made according to the method disclosed in U.S. Pat. No. 4,676,980 (Segel et al.)

The preferred epitopic site of a mutant JAK3 kinase polypeptide specific antibody of the invention is a peptide fragment consisting essentially of about 11 to 17 amino acids of the human JAK3 kinase polypeptide sequence (SEQ ID NO: 1) which fragment encompasses the alanine-to-valine mutation at position 572 (see FIG. 1A). It will be appreciated that antibodies that specifically binding shorter or longer peptides/epitopes encompassing the mutated residue (A572V) of the mutant JAK3 kinase polypeptide are within the scope of the present invention.

The invention is not limited to use of antibodies, but includes equivalent molecules, such as protein binding domains or nucleic acid aptamers, which bind, in a mutant-protein or truncated-protein specific manner, to essentially the same epitope to which a mutant JAK3 kinase polypeptide-specific antibody useful in the methods of the invention binds. See, e.g., Neuberger et al., Nature 312: 604 (1984). Such equivalent non-antibody reagents may be suitably employed in the methods of the invention further described below.

Polyclonal antibodies useful in practicing the methods of the invention may be produced according to standard techniques by immunizing a suitable animal (e.g., rabbit, goat, etc.) with an antigen encompassing a desired mutant-protein specific epitope (e.g. the sequence comprising the A572V mutation site) collecting immune serum from the animal, and separating the polyclonal antibodies from the immune serum, and purifying polyclonal antibodies having the desired specificity, in accordance with known procedures. The antigen may be a synthetic peptide antigen comprising the desired epitopic sequence, selected and constructed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201:264-283 (1991); Merrifield, J. Am. Chem. Soc. 85: 21-49 (1962)). Polyclonal antibodies produced as described herein may be screened and isolated as further described below.

Monoclonal antibodies may also be beneficially employed in the methods of the invention, and may be produced in hybridoma cell lines according to the well-known technique of Kohler and Milstein. Nature 265:495-97 (1975); Kohler and Milstein, Eur. J. Immunol. 6: 511 (1976); see also, CURRENT PROTOCOLS IN MOLECULAR BIOLOGY, Ausubel et al., Eds. (1989). Monoclonal antibodies so produced are highly specific, and improve the selectivity and specificity of assay methods provided by the invention. For example, a solution containing the appropriate antigen (e.g. a synthetic peptide comprising the mutant junction of mutant JAK3 kinase polypeptide) may be injected into a mouse and, after a sufficient time (in keeping with conventional techniques), the mouse sacrificed and spleen cells obtained. The spleen cells are then immortalized by fusing them with myeloma cells, typically in the presence of polyethylene glycol, to produce hybridoma cells. Rabbit mutant hybridomas, for example, may be produced as described in U.S. Pat. No. 5,675,063, K. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246:1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial mutant, or prepared secondarily, from a parental hybridoma secreting a monoclonal antibody of different isotype by using the sib selection technique to isolate class-switch variants (Steplewski, et al., Proc. Nat'l. Acad. Sci., 82:8653 (1985); Spira et al., J. Immunol. Methods, 74.307 (1984)). The antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Further still, U.S. Pat. No. 5,194,392, Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, this method involves detecting or determining a sequence of monomers which is a topographical equivalent of a ligand which is complementary to the ligand binding site of a particular receptor of interest. Similarly, U.S. Pat. No. 5,480,971, Houghten et al., (1996) discloses linear C₁-C-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

Antibodies useful in the methods of the invention, whether polyclonal or monoclonal, may be screened for epitope and mutant protein specificity according to standard techniques. See, e.g. Czernik et al., Methods in Enzymology, 201: 264-283 (1991). For example, the antibodies may be screened against a peptide library by ELISA to ensure specificity for both the desired antigen and, if desired, for reactivity only with a mutant JAK3 kinase polypeptide of the invention and not with wild type JAK3. The antibodies may also be tested by Western blotting against cell preparations containing target protein to confirm reactivity with the only the desired target and to ensure no appreciable binding to other JAK3 mutants not containing the A572V point mutation. The production, screening, and use of mutant protein-specific antibodies is known to those of skill in the art, and has been described.

Mutant polypeptide-specific antibodies useful in the methods of the invention may exhibit some limited cross-reactivity with similar epitopes in other highly homologous proteins. This is not unexpected as most antibodies exhibit some degree of cross-reactivity, and anti-peptide antibodies will often cross-react with epitopes having high homology or identity to the immunizing peptide. See, e.g., Czernik, supra. Gross-reactivity with other mutant proteins is readily characterized by Western blotting alongside markers of known molecular weight. Amino acid sequences of cross-reacting proteins may be examined to identify sites highly homologous or identical to the mutant JAK3 kinase polypeptide sequence to which the antibody binds. Undesirable cross-reactivity can be removed by negative selection using antibody purification on peptide columns (e.g. selecting out antibodies that bind either wild type JAK3 or which bind highly homologous sequences on different proteins).

Mutant JAK3 kinase polypeptide-specific antibodies of the invention that are useful in practicing the methods disclosed herein are ideally specific for human mutant polypeptide, but are not limited only to binding the human species, per se. The invention includes the production and use of antibodies that also bind conserved and highly homologous or identical epitopes in other mammalian species (e.g. mouse, rat, monkey). Highly homologous or identical sequences in other species can readily be identified by standard sequence comparisons, such as using BLAST, with the human mutant JAK3 kinase polypeptide sequence disclosed herein (SEQ ID NO: 1). The proteins sequences of JAK3 kinase from other mammalian species (mouse, rat, pig, cat) have been published and are available in the SwissProt database.

Antibodies employed in the methods of the invention may be further characterized by, and validated for, use in a particular assay format, for example FC, IHC, and/or ICC. The use of mutant JAK3 kinase polypeptide-specific antibodies in such methods is further described in Section F below. Antibodies may also be advantageously conjugated to fluorescent dyes (e.g. Alexa488, PE), or labels such as quantum dots, for use in multi-parametric analyses along with other signal transduction (phospho-AKT, phospho-Erk ½) and/or cell marker (cytokeratin) antibodies, as further described in Section F below.

In practicing the methods of the invention, the expression and/or activity of wild type JAK3 kinase in a given biological sample may also be advantageously examined using antibodies (either phospho-specific or total) for these wild type proteins. Such antibodies may also be produced according to standard methods, as described above. The amino acid sequences of human JAK3 is published (see FIG. 1A and referenced SwissProt Accession No.), as are the sequences of this proteins from other species, including mammalian, as noted above.

Detection of wild type JAK3 expression and/or activation, along with mutant JAK3 kinase polypeptide expression, in a biological sample (e.g. a tumor sample) can provide information on whether the mutant protein alone is driving the tumor, or whether wild type JAK3 is also activated and driving the tumor. Such information is clinically useful in assessing whether targeting the mutant protein or the wild type protein(s), or both, or is likely to be most beneficial in inhibiting progression of the tumor, and in selecting an appropriate therapeutic or combination thereof.

It will be understood that more than one antibody may be used in the practice of the above-described methods. For example, one or more mutant JAK3 kinase polypeptide-specific antibodies together with one or more antibodies specific for another kinase (such as JAK1 kinase), receptor, or kinase substrate (such as STAT5) that is suspected of being, or potentially is, activated in a cancer in which mutant JAK3 kinase polypeptide is expressed may be simultaneously employed to detect the activity of such other signaling molecules in a biological sample comprising cells from such cancer.

Those of skill in the art will appreciate that mutant JAK3 kinase polypeptides of the present invention and the mutant junction epitope-bearing fragments thereof described above can be combined with parts of the constant domain of immunoglobulins (IgG), resulting in chimeric polypeptides. These mutant proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331: 84-86 (1988)). Mutant proteins that have a disulfide-linked dimeric structure due to the IgG part can also be more efficient in binding and neutralizing other molecules than the monomeric mutant JAK3 kinase polypeptide alone (Fountoulakis et al., J Biochem 270: 3958-3964 (1995)).

### Heavy-Isotope Labeled Peptides (AQUA Peptides).

Mutant JAK3 kinase polypeptide-specific reagents useful in the practice of the disclosed methods may also comprise heavy-isotope labeled peptides suitable for the absolute quantification of expressed mutant JAK3 kinase polypeptide in a biological sample. The production and use of AQUA peptides for the absolute quantification of proteins (AQUA) in complex mixtures has been described. See WO/03016861, “Absolute Quantification of Proteins and Modified Forms Thereof by Multistage Mass Spectrometry,” Gygi et al. and also Gerber et al., Proc. Natl. Aced. Sci. U.S.A. 100: 6940-5 (2003) (the teachings of which are hereby incorporated herein by reference, in their entirety).

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein phosphorylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a 7-Da mass shift. The newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al. supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g. trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures.

Since an absolute amount of the AQUA peptide is added (e.g. 250 fmol), the ratio of the areas under the curve can be used to determine the precise expression levels of a protein or phosphorylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known sequence previously identified by the IAP-LC-MS/MS method within in a target protein. If the site is modified, one AQUA peptide incorporating the modified form of the particular residue within the site may be developed, and a second AQUA peptide incorporating the unmodified form of the residue developed. In this way, the two standards may be used to detect and quantify both the modified an unmodified forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g. trypsin, hepsin), metallo proteases (e.g. PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, peptides longer than about 20 amino acids are not preferred. The preferred ranged is about 7 to 15 amino acids. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e. to determine what fraction of the total amount of protein is represented by the modified form). For example, peptide standards for both the phosphorylated and unphosphorylated form of a protein known to be phosphorylated at a particular site can be used to quantify the amount of phosphorylated form in a sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: The mass should be unique to shift fragments masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ²H, ¹³C, ¹⁵N, ¹⁷O, ¹⁸O, or ³⁴S, are among preferred labels. Pairs of peptide internal standards that incorporate a different isotope label may also be prepared. Preferred amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature is that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably employed. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g. by HPLC, reverse-phase HPLC, capillary electrophoresis, ion exchange chromatography, etc.) to isolate the labeled internal standard and its corresponding target peptide from other peptides in the sample. Microcapillary LC is a preferred method.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

AQUA internal peptide standards (heavy-isotope labeled peptides) may desirably be produced, as described above, to detect and quantify any unique site (e.g. the A572V mutation point) within a mutant JAK3 polypeptide of the invention. For example, an AQUA phosphopeptide may be prepared that corresponds to the JAK3 peptide sequence immediately encompassing the A572V mutation point (see FIG. 1A). Peptide standards may be produced and such standards employed in the AQUA methodology to detect and quantify the presence of mutant (A572V) JAK3 kinase (i.e. the presence of the peptide sequence encompassing the A572V point mutation) in a biological sample.

For example, an exemplary AQUA peptide of the invention comprises the amino acid sequence EVA, which corresponds to the three amino acids immediately flanking each side of the A572V mutation point in the mutant JAK3 kinase polypeptide (see FIG. 1A). It will be appreciated that larger AQUA peptides comprising the mutant junction sequence (and additional residues downstream or upstream of it) may also be constructed. Similarly, a smaller AQUA peptide comprising less than all of the residues of such sequence (but still comprising the point of mutant junction itself) may alternatively be constructed. Such larger or shorter AQUA peptides are within the scope of the present invention, and the selection and production of preferred AQUA peptides may be carried out as described above (see Gygi et al., Gerber et al., supra.).

### Nucleic Acid Probes.

Mutant-specific reagents provided by the invention also include nucleic acid probes and primers suitable for detection of a mutant JAK3 kinase polynucleotide, as described in detail in Section B above. The specific use of such probes in assays such as fluorescence in-situ hybridization (FISH) or polymerase chain reaction (PCR) amplification is described in Section F below.

Also provided by the invention is a kit for the detection of mutant (A572V) JAK3 kinase in a biological sample, the kit comprising an isolated mutant JAK3 kinase-specific reagent of the invention and one or more secondary reagents. Suitable secondary reagents for employment in a kit are familiar to those of skill in the art, and include, by way of example, buffers, detectable secondary antibodies or probes, kinases, activating agents, kinase substrates, and the like.

### F. Diagnostic Applications & Assay Formats.

The methods of the invention may be carried out in a variety of different assay formats known to those of skill in the art.

### Immunoassays.

Immunoassays useful in the practice of the methods of the invention may be homogenous immunoassays or heterogeneous immunoassays. In a homogeneous assay the immunological reaction usually involves a mutant JAK3 kinase polypeptide-specific reagent (e.g. a mutant JAK3 kinase polypeptide-specific antibody), a labeled analyte, and the biological sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radio-isotopes, fluorescent dyes, enzymes, bacteriophages, coenzymes, and so forth. Semi-conductor nanocrystal labels, or “quantum dots”, may also be advantageously employed, and their preparation and use has been well described. See generally, K. Barovsky, Nanotech. Law & Bus. 1(2): Article 14 (2004) and patents cited therein.

In a heterogeneous assay approach, the reagents are usually the biological sample, a mutant JAK3 kinase polypeptide-specific reagent (e.g., a mutant JAK3 kinase-specific antibody), and suitable means for producing a detectable signal. Biological samples as further described below may be used. The antibody is generally immobilized on a support, such as a bead, plate or slide, and contacted with the sample suspected of containing the antigen in a liquid phase. The support is then separated from the liquid phase and either the support phase or the liquid phase is examined for a detectable signal employing means for producing such signal. The signal is related to the presence of the analyte in the biological sample. Means for producing a detectable signal include the use of radioactive labels, fluorescent labels, enzyme labels, quantum dots, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof, which may be useful for carrying out the methods disclosed herein, are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well known to those of skill in the art. See id. Mutant JAK3 kinase polypeptide-specific monoclonal antibodies may be used in a “two-site” or “sandwich” assay, with a single hybridoma cell line serving as a source for both the labeled monoclonal antibody and the bound monoclonal antibody. Such assays are described in U.S. Pat. No. 4,376,110. The concentration of detectable reagent should be sufficient such that the binding of mutant JAK3 kinase polypeptide is detectable compared to background.

Antibodies useful in the practice of the methods disclosed herein may be conjugated to a solid support suitable for a diagnostic assay (e.g., beads, plates, slides or wells formed from materials such as latex or polystyrene) in accordance with known techniques, such as precipitation. Antibodies or other mutant JAK3 kinase polypeptide-binding reagents may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Cell-based assays, such flow cytometry (FC), immuno-histochemistry (IHC), or immunofluorescence (IF) are particularly desirable in practicing the methods of the invention, since such assay formats are clinically-suitable, allow the detection of mutant JAK3 kinase polypeptide expression in vivo, and avoid the risk of artifact changes in activity resulting from manipulating cells obtained from, e.g. a tumor sample in order to obtain extracts. Accordingly, in some preferred embodiment, the methods of the invention are implemented in a flow-cytometry (FC), immuno-histochemistry (IHC), or immunofluorescence (IF) assay format.

Flow cytometry (FC) may be employed to determine the expression of mutant JAK3 kinase polypeptide in a mammalian leukemia sample before, during, and after treatment with a drug targeted at inhibiting JAK3 kinase activity. For example, tumor cells from a bone marrow sample may be analyzed by flow cytometry for mutant JAK3 kinase polypeptide expression and/or activation, as well as for markers identifying cancer cell types, etc., if so desired. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 2% paraformaldehyde for 10 minutes at 37° C. followed by permeabilization in 90% methanol for 30 minutes on ice. Cells may then be stained with the primary mutant JAK3 kinase polypeptide-specific antibody, washed and labeled with a fluorescent-labeled secondary antibody. The cells would then be analyzed on a flow cytometer (e.g. a Beckman Coulter FC500) according to the specific protocols of the instrument used. Such an analysis would identify the level of expressed mutant JAK3 kinase polypeptide in the tumor. Similar analysis after treatment of the tumor with a JAK3-inhibiting therapeutic would reveal the responsiveness of a mutant JAK3 kinase polypeptide-expressing tumor to the targeted inhibitor of JAK3 kinase.

Immunohistochemical (IHC) staining may be also employed to determine the expression and/or activation status of mutant JAK3 kinase polypeptide in a mammalian cancer (e.g. AML) before, during, and after treatment with a drug targeted at inhibiting JAK3 kinase activity. IHC may be carried out according to well-known techniques. See, e.g., ANTIBODIES; A LABORATORY MANUAL, Chapter 10, Harlow & Lane Eds., Cold Spring Harbor Laboratory (1988). Briefly, and by way of example, paraffin-embedded tissue (e.g. tumor tissue from a biopsy) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary anti-mutant JAK3 kinase polypeptide antibody and secondary antibody; and finally detecting using ABC avidin/biotin method according to manufacturer's instructions.

Immunofluorescence (IF) assays may be also employed to determine the expression and/or activation status of mutant JAK3 kinase polypeptide in a mammalian cancer before, during, and after treatment with a drug targeted at inhibiting JAK3 kinase activity. IF may be carried out according to well-known techniques. See, e.g., J. M. Polak and S. Van Noorden (1997) INTRODUCTION TO IMMUNOCYTOCHEMISTRY, 2nd Ed.; ROYAL MICROSCOPY SOCIETY MICROSCOPY HANDBOOK 37, BioScientific/Springer-Verlag. Briefly, and by way of example, patient samples may be fixed in paraformaldehyde followed by methanol, blocked with a blocking solution such as horse serum, incubated with the primary antibody against mutant JAK3 kinase polypeptide followed by a secondary antibody labeled with a fluorescent dye such as Alexa 488 and analyzed with an epifluorescent microscope.

Antibodies employed in the above-described assays may be advantageously conjugated to fluorescent dyes (e.g. Alexa488, PE), or other labels, such as quantum dots, for use in multi-parametric analyses along with other signal transduction (EGFR, phospho-AKT, phospho-Erk ½) and/or cell marker (cytokeratin) antibodies.

A variety of other protocols, including enzyme-linked immunosorbent assay (ELISA), radio-Immunoassay (RIA), and fluorescent-activated cell sorting (FACS), for measuring mutant JAK3 kinase polypeptide are known in the art and provide a basis for diagnosing altered or abnormal levels of mutant JAK3 kinase polypeptide expression. Normal or standard values for mutant JAK3 kinase polypeptide expression are established by combining body fluids or cell extracts taken from normal mammalian subjects, preferably human, with antibody to mutant JAK3 kinase polypeptide under conditions suitable for complex formation. The amount of standard complex formation may be quantified by various methods, but preferably by photometric means. Quantities of mutant JAK3 kinase polypeptide expressed in subject, control, and disease samples from biopsied tissues are compared with the standard values. Deviation between standard and subject values establishes the parameters for diagnosing disease.

### Peptide & Nucleic Acid Assays.

Similarly, AQUA peptides for the detection/quantification of expressed mutant JAK3 kinase polypeptide in a biological sample comprising cells from a tumor may be prepared and used in standard AQUA assays, as described in detail in Section E above. Accordingly, in some preferred embodiments of the methods of the invention, the mutant JAK3 kinase polypeptide-specific reagent comprises a heavy isotope labeled phosphopeptide (AQUA peptide) corresponding to a peptide sequence comprising the mutant junction of mutant JAK3 kinase polypeptide, as described above in Section E.

Mutant JAK3 polypeptide-specific reagents useful in practicing the methods of the invention may also be mRNA, oligonucleotide or DNA probes that can directly hybridize to, and detect, mutant or truncated polypeptide expression transcripts in a biological sample. Such probes are discussed in detail in Section B above. Briefly, and by way of example, formalin-fixed, paraffin-embedded patient samples may be probed with a fluorescein-labeled RNA probe followed by washes with formamide, SSC and PBS and analysis with a fluorescent microscope.

Polynucleotides encoding mutant JAK3 kinase polypeptide may also be used for diagnostic purposes. The polynucleotides that may be used include oligonucleotide sequences, antisense RNA and DNA molecules. The polynucleotides may be used to detect and quantitate gene expression in biopsied tissues in which expression of mutant JAK3 kinase polypeptide or truncated active JAK3 kinase polypeptide may be correlated with disease. For example, the diagnostic assay may be used to distinguish between absence, presence, and excess expression of mutant JAK3 kinase polypeptide, and to monitor regulation of mutant JAK3 kinase polypeptide levels during therapeutic intervention.

In one preferred embodiment, hybridization with PCR probes which are capable of detecting polynucleotide sequences, including genomic sequences, encoding mutant JAK3 kinase polypeptide or truncated active JAK3 kinase polypeptide, or closely related molecules, may be used to identify nucleic acid sequences which encode mutant JAK3 polypeptide. The construction and use of such probes is described in Section B above. The specificity of the probe, whether it is made from a highly specific region, e.g., 10 unique nucleotides in the mutant junction, or a less specific region, e.g., the 3′ coding region, and the stringency of the hybridization or amplification (maximal, high, intermediate, or low) will determine whether the probe identifies only naturally occurring sequences encoding mutant JAK3 polypeptide, alleles, or related sequences.

Probes may also be used for the detection of related sequences, and should preferably contain at least 50% of the nucleotides from any of the mutant JAK3 polypeptide encoding sequences. The hybridization probes of the subject invention may be DNA or RNA and derived from the nucleotide sequence of SEQ ID NO: 2 and encompassing the A572V mutation point (see FIGS. 1A-1B), or from genomic sequence including promoter, enhancer elements, and introns of the naturally occurring JAK3 polypeptides but comprising the A572 mutation point sequence, as further described in Section B above.

A mutant JAK3 kinase polynucleotide or truncated JAK3 polynucleotide of the invention may be used in Southern or northern analysis, dot blot, or other membrane-based technologies; in PCR technologies; or in dip stick, pin, ELISA or chip assays utilizing fluids or tissues from patient biopsies to detect altered JAK3 polypeptide expression. Such qualitative or quantitative methods are well known in the art. In a particular aspect, the nucleotide sequences encoding a mutant JAK3 polypeptide of the invention may be useful in assays that detect activation or induction of various cancers, including leukemias Mutant JAK3 polynucleotides may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable control sample, the nucleotide sequences have hybridized with nucleotide sequences in the sample, and the presence of altered levels of nucleotide sequences encoding mutant JAK3 kinase polypeptide in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease characterized by expression of mutant JAK3 kinase polypeptide, a normal or standard profile for expression is established. This may be accomplished by combining body fluids or cell extracts taken from normal subjects, either animal or human, with a sequence, or a fragment thereof, which encodes mutant JAK3 kinase polypeptide, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease.

Once disease is established and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months.

Additional diagnostic uses for mutant JAK3 polynucleotides of the invention may involve the use of polymerase chain reaction (PCR), a preferred assay format that is standard to those of skill in the art. See, e.g., MOLECULAR CLONING, A LABORATORY MANUAL, 2nd. edition, Sambrook, J., Fritsch, E. F. and Maniatis, T., eds., Cold Spring Harbor Laboratory Press, Cold Spring Harbor, N.Y. (1989). PCR oligomers may be chemically synthesized, generated enzymatically, or produced from a recombinant source. Oligomers will preferably consist of two nucleotide sequences, one with sense orientation (5′ to 3′) and another with antisense (3′ to 5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Methods which may also be used to quantitate the expression of mutant JAK3 kinase polypeptide include radiolabeling or biotinylating nucleotides, coamplification of a control nucleic acid, and standard curves onto which the experimental results are interpolated (Melby et al., J. Immunol. Methods, 159:235-244 (1993); Duplaa et al. Anal. Biochem. 229-236 (1993)). The speed of quantitation of multiple samples may be accelerated by running the assay in an ELISA format where the oligomer of interest is presented in various dilutions and a spectrophotometric or calorimetric response gives rapid quantitation.

Other suitable methods for nucleic acid detection, such as minor groove-binding conjugated oligonucleotide probes (see, e.g. U.S. Pat. No. 6,951,930, “Hybridization-Triggered Fluorescent Detection of Nucleic Acids”) are known to those of skill in the art.

### Biological Samples.

Biological samples useful in the practice of the methods of the invention may be obtained from any mammal in which a cancer characterized by the expression of a mutant (A572V) JAK3 kinase polypeptide is present or developing. In one embodiment, the mammal is a human, and the human may be a candidate for a JAK3-inhibiting therapeutic for the treatment of a leukemia, e.g. AML. The human candidate may be a patient currently being treated with, or considered for treatment with, a JAK3 kinase inhibitor, such as CP-690,550 (Pfizer, Inc.). In another embodiment, the mammal is large animal, such as a horse or cow, while in other embodiments, the mammal is a small animal, such as a dog or cat, all of which are known to develop cancers, including leukemias.

Any biological sample comprising cells (or extracts of cells) from a mammalian cancer is suitable for use in the methods of the invention. Serum and bone marrow samples may be particularly preferred for patients with leukemia, and may be obtained by standard methods. Circulating tumor cells may also be obtained from serum using tumor markers, cytokeratin protein markers or other methods of negative selection as described (see Ma et al., Anticancer Res. 23(1A): 49-62 (2003)). For cancers involving solid tumors, the biological sample may comprise cells obtained from a tumor biopsy, which maybe be obtained according to standard clinical techniques. For example, aberrant expression/activity of JAK3 has been observed in a spectrum of cancers including mantle cell lymphoma, Hodgkin disease and colon cancer. See, e.g., Yared et al., supra.; Lin et al., supra.

A biological sample may comprise cells (or cell extracts) from a cancer in which mutant JAK3 kinase polypeptide is expressed and/or activated but wild type JAK3 kinase is not. Alternatively, the sample may comprise cells from a cancer in which both the mutant JAK3 polypeptide and wild type JAK3 kinase are expressed and/or activated, or in which wild type JAK3 kinase is expressed and/or active, but mutant JAK3 polypeptide is not.

Cellular extracts of the foregoing biological samples may be prepared, either crude or partially (or entirely) purified, in accordance with standard techniques, and used in the methods of the invention. Alternatively, biological samples comprising whole cells may be utilized in preferred assay formats such as immunohistochemistry (IHC), flow cytometry (FC), and immunofluorescence (IF), as further described above. Such whole-cell assays are advantageous in that they minimize manipulation of the tumor cell sample and thus reduce the risks of altering the in vivo signaling/activation state of the cells and/or introducing artifact signals. Whole cell assays are also advantageous because they characterize expression and signaling only in tumor cells, rather than a mixture of tumor and normal cells.

In practicing the disclosed method for determining whether a compound inhibits progression of a tumor characterized by a mutant JAK3 polypeptide or polynucleotide, biological samples comprising cells from mammalian bone marrow transplant models or xenografts may also be advantageously employed. Preferred xenografts (or transplant recipients) are small mammals, such as mice, harboring human tumors (or leukemias) that express a mutant JAK3 kinase polypeptide. Xenografts harboring human tumors are well known in the art (see Kal, Cancer Treat Res. 72: 155-69 (1995)) and the production of mammalian xenografts harboring human tumors is well described (see Winograd et al., In Vivo. 1(1): 1-13 (1987)). Similarly the generation and use of bone marrow transplant models is well described (see, e.g., Schwaller, et al., EMBO J. 17: 5321-333 (1998); Kelly et al., Blood 99: 310-318 (2002)). By “cancer characterized by” a mutant JAK3 polynucleotide or polypeptide is meant a cancer in which such mutant (A572V) JAK3 gene and/or expressed polypeptide are present, as compared to a cancer in which such mutated gene and/or polypeptide are not present.

In assessing mutant JAK3 polynucleotide presence or polypeptide expression in a biological sample comprising cells from a mammalian cancer tumor, a control sample representing a cell in which such translocation and/or mutant protein do not occur may desirably be employed for comparative purposes. Ideally, the control sample comprises cells from a subset of the particular cancer (e.g. leukemia) that is representative of the subset in which the mutation (i.e. the A572V point mutation) does not occur and/or the mutant polypeptide is not expressed. Comparing the level in the control sample versus the test biological sample thus identifies whether the mutant JAK3 polynucleotide and/or polypeptide is/are present. Alternatively, since mutant JAK3 kinase polynucleotide and/or polypeptide, or truncated JAK3 polynucleotide and/or polypeptide, may not be present in the majority of cancers, any tissue that similarly does not express such mutant JAK3 polypeptide (or harbor the mutant polynucleotide) may be employed as a control.

The methods described below will have valuable diagnostic utility for cancers characterized by mutant (A572V) JAK3 polynucleotide and/or polypeptide, and treatment decisions pertaining to the same. For example, biological samples may be obtained from a subject that has not been previously diagnosed as having a cancer characterized by a mutant JAK3 polypeptide, nor has yet undergone treatment for such cancer, and the method is employed to diagnostically identify a tumor in such subject as belonging to a subset of tumors (e.g. leukemias) in which mutant JAK3 kinase polynucleotide and/or polypeptide is present and/or expressed. The methods of the invention may also be employed to monitor the progression or inhibition of a mutant JAK3 kinase polypeptide-expressing cancer following treatment of a subject with a composition comprising a JAK3 kinase-inhibiting therapeutic or combination of therapeutics.

Such diagnostic assay may be carried out subsequent to or prior to preliminary evaluation or surgical surveillance procedures. The identification method of the invention may be advantageously employed as a diagnostic to identify patients having cancer, such as AML, driven by the mutant JAK3 kinase, which patients would be most likely to respond to therapeutics targeted at inhibiting JAK3 kinase activity, such as CP-690,550, or its analogues. The ability to select such patients would also be useful in the clinical evaluation of efficacy of future JAK3-targeted therapeutics as well as in the future prescription of such drugs to patients.

### Diagnostics.

The ability to selectively identify cancers in which a mutant (A572V) JAK3 polynucleotide or polypeptide is/are present enables important new methods for accurately identifying such tumors for diagnostic purposes, as well as obtaining information useful in determining whether such a tumor is likely to respond to a JAK3-inhibiting therapeutic composition, or likely to be partially or wholly non-responsive to an inhibitor targeting a different kinase when administered as a single agent for the treatment of the cancer.

Accordingly, in one embodiment, the invention provides a method for detecting the presence of a mutant JAK3 polynucleotide and/or polypeptide in a cancer, the method comprising the steps of:

(a) obtaining a biological sample from a patient having cancer; and

(b) utilizing at least one reagent that detects a mutant JAK3 polynucleotide or polypeptide of the invention to determine whether a mutant (A572V) JAK3 polynucleotide or polypeptide is/are present in the biological sample.

In one preferred embodiment, the cancer is a leukemia, such as acute myelogenous leukemia (AM L). In still other preferred embodiments, the presence of a mutant JAK3 polypeptide identifies a cancer that is likely to respond to a composition comprising at least one JAK3 kinase-inhibiting therapeutic. Exemplary JAK3-inhibiting therapeutics include, but are not limited to, CP-690,550 (Pfizer, Inc.), WP-1034, and their analogues. JAK3-inhibiting therapeutics are further described in Section G below.

In some preferred embodiments, the diagnostic methods of the invention are implemented in a flow-cytometry (FC), immuno-histochemistry (IHC), or immuno-fluorescence (IF) assay format, as described above. In another preferred embodiment, the activity of the mutant JAK3 kinase polypeptide is detected. In other preferred embodiments, the diagnostic methods of the invention are implemented in a polymerase chain reaction (PCR) sequencing assay format, as described above.

The invention further provides a method for determining whether a compound inhibits the progression of a cancer characterized by a mutant (A572V) JAK3 kinase polynucleotide and/or polypeptide, said method comprising the step of determining whether said compound inhibits the expression and/or activity of said mutant JAK3 kinase polypeptide in said cancer. In one preferred embodiment, inhibition of expression and/or activity of the mutant JAK3 kinase polypeptide is determined using at least one reagent that specifically detects a mutant JAK3 kinase polynucleotide or polypeptide of the invention. Compounds suitable for inhibition of JAK3 kinase activity are discussed in more detail in Section G below.

Mutant JAK3 polynucleotide probes and polypeptide-specific reagents useful in the practice of the methods of the invention are described in further detail in sections B and D above. In one preferred embodiment, the mutant JAK3 kinase polypeptide-specific reagent comprises a mutant polypeptide-specific antibody. In another preferred embodiment, the mutant polypeptide-specific reagent comprises a heavy-isotope labeled phosphopeptide (AQUA peptide) corresponding to the mutation site (A572V) sequence of mutant JAK3 kinase polypeptide (see FIG. 1A).

The methods of the invention described above may also optionally comprise the step of determining the level of expression or activation of other kinases, such as wild type JAK3 and JAK1, or other downstream signaling molecules, such as STAT5, in said biological sample. Profiling both mutant JAK3 kinase polypeptide expression/activation and expression/activation of other kinases and pathways in a given biological sample can provide valuable information on which kinase(s) and pathway(s) is/are driving the disease, and which therapeutic regime is therefore likely to be of most benefit.

### Compound Screening.

The discovery of the novel mutant JAK3 kinase polypeptide described herein also enables the development of new compounds that inhibit the activity of these mutant JAK3 proteins. Accordingly, the invention also provides, in part, a method for determining whether a compound inhibits the progression of a cancer characterized by a mutant (A572V) JAK3 kinase polynucleotide and/or polypeptide, said method comprising the step of determining whether said compound inhibits the expression and/or activity of said mutant JAK3 kinase polypeptide in said cancer. In one preferred embodiment, inhibition of expression and/or activity of the mutant JAK3 kinase polypeptide or truncated active JAK3 kinase polypeptide is determined using at least one reagent that detects a mutant JAK3 polynucleotide and/or mutant JAK3 polypeptide of the invention. Preferred reagents of the invention have been described above. Compounds suitable for the inhibition of JAK3 kinase activity are described in more detail in Section G below.

The compound may, for example, be a kinase inhibitor, such as a small molecule or antibody inhibitor. It may be a pan-kinase inhibitor, such as JAK I, with activity against several different kinases, or a kinase-specific inhibition, as further described below. Patient biological samples may be taken before and after treatment with the inhibitor and then analyzed, using methods described above, for the biological effect of the inhibitor on JAK3 kinase activity, including the phosphorylation of downstream substrate proteins, like STAT5. Such a pharmacodynamic assay may be useful in determining the biologically active dose of the drug that may be preferable to a maximal tolerable dose. Such information would also be useful in submissions for drug approval by demonstrating the mechanism of drug action. Identifying compounds with such desired inhibitory characteristics is further described in Section G below.

### G. Therapeutic Inhibition of Cancers.

In accordance with the present invention, it has now been shown that the progression of a mammalian cancer (AML) in which mutant (A572V) JAK3 kinase is expressed may be inhibited, in vivo, by inhibiting the activity of the mutant JAK3 kinase in such cancer. JAK3 activity in cancers characterized by expression of a mutant JAK3 polypeptide may be inhibited by contacting the cancer (e.g. a tumor) with a JAK3 kinase-inhibiting therapeutic, such as a small-molecule kinase inhibitor like CP-690,550. As further described in Example 3 below, growth inhibition of mutant JAK3 kinase protein-expressing leukemias, for example, can be accomplished by inhibiting this mutant kinase using an exemplary pan-JAK kinase-inhibiting therapeutic, JAK I, or by exemplary siRNA silencing. Accordingly, the invention provides, in part, a method for inhibiting the progression of a cancer that expresses mutant (A572V) JAK3 kinase polypeptide by inhibiting the expression and/or activity of the mutant JAK3 kinase(s) in the cancer.

A JAK3 kinase-inhibiting therapeutic may be any composition comprising at least one compound, biological or chemical, which inhibits, directly or indirectly, the expression and/or activity of JAK3 kinase in vivo, including the exemplary classes of compounds described below. Such compounds include therapeutics that act directly on JAK3 kinase itself, or on proteins or molecules that modify the activity of JAK3, or that act indirectly by inhibiting the expression of JAK3. Such compositions also include compositions comprising only a single JAK3 kinase inhibiting compound, as well as compositions comprising multiple therapeutics (including those against other RTKs), which may also include a non-specific therapeutic agent like a chemotherapeutic agent or general transcription inhibitor.

### Small-Molecule Inhibitors.

In some preferred embodiments, a JAK3-inhibiting therapeutic useful in the practice of the methods of the invention is a targeted, small molecule inhibitor, such as CP-690,550 (Pfizer, Inc.), and its analogues. For example, as presently shown (see Example 3), administration of CP-690,550 to a human AML cell line (CMK) expressing the mutant (A572V) JAK3 kinase protein selectively inhibited the growth and survival of those cancer cells, but not in control cells that do not express the mutant JAK3 protein. CP-690,550, which specifically binds to and blocks the ATP-binding site of JAK3 kinase, thereby preventing phosphorylation and activation of this enzyme, is commercially available and some of its properties have been described. See, e.g. Borne et al., Tranplantation 80 (12): 1756-1764 (2005); Paniagua et al., Tranplantation 80 (9): 1283-1292 (2005). Thus, CP-690,550 is an exemplary small molecule inhibitor that should be considered for patients with a cancer characterized by the mutant (A572V) JAK3 kinase.

Other preferred small-molecule inhibitors of JAK3 include the JAK-STAT inhibitor, WP-1034 and the pan-JAK inhibitor, JAK I. These compounds are commercially available and their JAK3 kinase inhibitory properties have been described. See, e.g. Faderl et al., Anticancer Res. 25 (3B): 1841-1850 (2005).

Small molecule targeted inhibitors are a class of molecules that typically inhibit the activity of their target enzyme by specifically, and often irreversibly, binding to the catalytic site of the enzyme, and/or binding to an ATP-binding cleft or other binding site within the enzyme that prevents the enzyme from adopting a conformation necessary for its activity. Small molecule inhibitors may be rationally designed using X-ray crystallographic or computer modeling of JAK3 kinase three-dimensional structure, or may found by high throughput screening of compound libraries for inhibition of JAK3. Such methods are well known in the art, and have been described. Specificity of JAK3 inhibition may be confirmed, for example, by examining the ability of such compound to inhibit JAK3 activity, but not other kinase activity, in a panel of kinases, and/or by examining the inhibition of JAK3 activity in a biological sample comprising leukemia cells, as described above. Such screening methods are further described below.

### Indirect Inhibitors.

JAK3-inhibiting compounds useful in the practice of the disclosed methods may also be compounds that indirectly inhibit JAK3 activity by inhibiting the activity of proteins or molecules other than JAK3 kinase itself. Such inhibiting therapeutics may be targeted inhibitors that modulate the activity of key regulatory kinases that phosphorylate or de-phosphorylate (and hence activate or deactivate) JAK3 itself, or interfere with binding of ligands. As with other kinases, JAK3 regulates downstream signaling through a network of adaptor proteins and downstream kinases. As a result, induction of cell growth and survival by JAK3 activity may be inhibited by targeting these interacting or downstream proteins.

JAK3 kinase activity may also be indirectly inhibited by using a compound that inhibits the binding of an activating molecule necessary for JAK3 to adopt its active conformation or for JAK3 to effect its signaling. For example, as disclosed herein, mutant (A572V) JAK3 kinase binds to and interacts with JAK1 kinase, which itself in turn then directly phosphorylates STAT5 downstream. Thus, by way of example, JAK1 can be targeted to prevent its interaction with activated mutant (A572V) JAK3 kinase. Indirect inhibitors of JAK3 activity may be rationally designed using X-ray crystallographic or computer modeling of JAK3 three dimensional structure, or may found by high throughput screening of compound libraries for inhibition of key upstream regulatory enzymes and/or necessary binding molecules, which results in inhibition of JAK3 kinase activity. Such approaches are well known in the art, and have been described. JAK3 inhibition by such therapeutics may be confirmed, for example, by examining the ability of the compound to inhibit JAK3 activity, but not other kinase activity, in a panel of kinases, and/or by examining the inhibition of JAK3 activity in a biological sample comprising cancer cells, e.g. AML cells, as described above. Methods for identifying compounds that inhibit a cancer characterized by a mutant JAK3 kinase polynucleotide and/or polypeptide are further described below. Anti-Sense and/or Transcription Inhibitors.

JAK3 inhibiting therapeutics may also comprise anti-sense and/or transcription inhibiting compounds that inhibit JAK3 kinase activity by blocking transcription of the gene encoding the mutant (A572V) JAK3 kinase gene (see FIG. 1B). For example, the inhibition of various receptor kinases, including VEGFR, EGFR, and IGFR, and FGFR, by antisense therapeutics for the treatment of cancer has been described. See, e.g., U.S. Pat. Nos. 6,734,017; 6,710,174, 6,617,162; 6,340,674; 5,783,683; 5,610,288.

Antisense oligonucleotides may be designed, constructed, and employed as therapeutic agents against target genes in accordance with known techniques. See, e.g. Cohen, J., Trends in Pharmacol. Sci. 10(11): 435-437 (1989); Marcus-Sekura, Anal. Biochem. 172: 289-295 (1988); Weintraub, H., Sci. AM. pp. 4046 (1990); Van Der Krol et al., BioTechniques 6(10): 958-976 (1988); Skorski et al., Proc. Natl. Acad. Sci. USA (1994) 91: 4504-44508. Inhibition of human carcinoma growth in vivo using an antisense RNA inhibitor of EGFR has recently been described. See U.S. Patent Publication No. 20040047847, “Inhibition of Human Squamous Cell Carcinoma Growth in vivo by Epidermal Growth Factor Receptor Antisense RNA Transcribed from a Pol III Promoter,” Mar. 11, 2004, He et al. Similarly, a JAK3-inhibiting therapeutic comprising at least one antisense oligonucleotide against a mammalian JAK3 gene (see FIG. 1B (SEQ ID NO: 2)) or mutant JAK3 kinase polynucleotide (same as wild type JAK3 but having the A572V point mutation) may be prepared according to methods described above. Pharmaceutical compositions comprising JAK3-inhibiting antisense compounds may be prepared and administered as further described below.

### Small Interfering RNA.

Small interfering RNA molecule (siRNA) compositions, which inhibit translation, and hence activity, of JAK3 through the process of RNA interference, may also be desirably employed in the methods of the invention. RNA interference, and the selective silencing of target protein expression by introduction of exogenous small double-stranded RNA molecules comprising sequence complimentary to mRNA encoding the target protein, has been well described. See, e.g. U.S. Patent Publication No. 20040038921, “Composition and Method for Inhibiting Expression of a Target Gene,” Feb. 26, 2004, Kreutzer et al.; U.S. Patent Publication No. 20020086356, “RNA Sequence-Specific Mediators of RNA Interference,” Jun. 12, 2003, Tuschl et al.,; U.S. Patent Publication 20040229266, “RNA Interference Mediating Small RNA Molecules,” Nov. 18, 2004, Tuschl et, al.

For example, as presently shown (see Example 3), siRNA-mediated silencing of expression of the mutant JAK3 kinase protein in a human leukemia cell line expressing the mutant protein selectively inhibited the growth, proliferation and survival of those cancer cells, but not in control cells that do not express the mutant JAK3 protein.

Double-stranded RNA molecules (dsRNA) have been shown to block gene expression in a highly conserved regulatory mechanism known as RNA interference (RNAi). Briefly, the RNAse III Dicer processes dsRNA into small interfering RNAs (siRNA) of approximately 22 nucleotides, which serve as guide sequences to induce target-specific mRNA cleavage by an RNA-induced silencing complex RISC (see Hammond et al., Nature (2000) 404: 293-296). RNAi involves a catalytic-type reaction whereby new siRNAs are generated through successive cleavage of longer dsRNA. Thus, unlike antisense, RNAi degrades target RNA in a non-stoichiometric manner. When administered to a cell or organism, exogenous dsRNA has been shown to direct the sequence-specific degradation of endogenous messenger RNA (mRNA) through RNAi.

A wide variety of target-specific siRNA products, including vectors and systems for their expression and use in mammalian cells, are now commercially available. See, e.g. Promega, Inc. (promega.com); Dharmacon, Inc. (dharmacon.com). Detailed technical manuals on the design, construction, and use of dsRNA for RNAi are available. See, e.g. Dharmacon's “RNAi Technical Reference & Application Guide”; Promega's “RNAi: A Guide to Gene Silencing.” JAK3-inhibiting siRNA products are also commercially available, and may be suitably employed in the method of the invention. See, e.g. Dharmacon, Inc., Lafayette, Colo. (Cat Nos. M-003162-03, MU-003162-03, D-003162-07 thru -10 (siGENOME™ SMARTselection and SMARTpool® siRNAs).

It has recently been established that small dsRNA less than 49 nucleotides in length, and preferably 19-25 nucleotides, comprising at least one sequence that is substantially identical to part of a target mRNA sequence, and which dsRNA optimally has at least one overhang of 14 nucleotides at an end, are most effective in mediating RNAi in mammals. See U.S. Patent Publication No. 20040038921, Kreutzer et al., supra; U.S. Patent Publication No. 20040229266, Tuschl et al., supra. The construction of such dsRNA, and their use in pharmaceutical preparations to silence expression of a target protein, in vivo, are described in detail in such publications.

If the sequence of the gene to be targeted in a mammal is known, 21-23 nt RNAs, for example, can be produced and tested for their ability to mediate RNAi in a mammalian cell, such as a human or other primate cell. Those 21-23 nt RNA molecules shown to mediate RNAi can be tested, if desired, in an appropriate animal model to further assess their in vivo effectiveness. Target sites that are known, for example target sites determined to be effective target sites based on studies with other nucleic acid molecules, for example ribozymes or antisense, or those targets known to be associated with a disease or condition such as those sites containing mutations or deletions, can be used to design siRNA molecules targeting those sites as well.

Alternatively, the sequences of effective dsRNA can be rationally designed/predicted screening the target mRNA of interest for target sites, for example by using a computer folding algorithm. The target sequence can be parsed in silica into a list of all fragments or subsequences of a particular length, for example 23 nucleotide fragments, using a custom Perl script or commercial sequence analysis programs such as Oligo, MacVector, or the GCG Wisconsin Package.

Various parameters can be used to determine which sites are the most suitable target sites within the target RNA sequence. These parameters include but are not limited to secondary or tertiary RNA structure, the nucleotide base composition of the target sequence, the degree of homology between various regions of the target sequence, or the relative position of the target sequence within the RNA transcript. Based on these determinations, any number of target sites within the RNA transcript can be chosen to screen siRNA molecules for efficacy, for example by using in vitro RNA cleavage assays, cell culture, or animal models. See, e.g., U.S. Patent Publication No. 20030170891, Sep. 11, 2003, McSwiggen J. An algorithm for identifying and selecting RNAi target sites has also recently been described. See U.S. Patent Publication No. 20040236517, “Selection of Target Sites for Antisense Attack of RNA,” Nov. 25, 2004, Drlica et al.

Commonly used gene transfer techniques include calcium phosphate, DEAE-dextran, electroporation and microinjection and viral methods (Graham et al. (1973) Virol 52: 456; McCutchan et al., (1968), J. Natl. Cancer Inst. 41: 351; Chu et al. (1987), Nucl. Acids Res. 15: 1311; raley et al., (1980), J. Biol. Chem. 255: 10431; Capecchi (1980), Cell 22: 479). DNA may also be introduced into cells using cationic liposomes (Feigner et al. (1987), Proc. Natl. Acad. Sci. USA 84: 7413). Commercially available cationic lipid formulations include Tfx 50 (Promega) or Lipofectamin 200 (Life Technologies). Alternatively, viral vectors may be employed to deliver dsRNA to a cell and mediate RNAi. See U.S Patent Publication No. 20040023390, “siRNA-mediated Gene Silencing with Viral Vectors,” Feb. 4, 2004, Davidson et al.

Transfection and vector/expression systems for RNAi in mammalian cells are commercially available and have been well described. See, e.g. Dharmacon, Inc., DharmaFECT™ system; Promega, Inc., siSTRIKE™ U6 Hairpin system; see also Gou et al. (2003) FEBS. 548, 113-118; Sui, G. et al. A DNA vector-based RNAi technology to suppress gene expression in mammalian cells (2002) Proc. Natl. Acad. Sci. 99, 5515-5520; Yu et al. (2002) Proc. Natl. Acad. Sci. 99, 6047-6052; Paul, C. et al. (2002) Nature Biotechnology 19, 505-508; McManus et al., (2002) RNA 8, 842-850.

siRNA interference in a mammal using prepared dsRNA molecules may then be effected by administering a pharmaceutical preparation comprising the dsRNA to the mammal. The pharmaceutical composition is administered in a dosage sufficient to inhibit expression of the target gene. dsRNA can typically be administered at a dosage of less than 5 mg dsRNA per kilogram body weight per day, and is sufficient to inhibit or completely suppress expression of the target gene. In general a suitable dose of dsRNA will be in the range of 0.01 to 2.5 milligrams per kilogram body weight of the recipient per day, preferably in the range of 0.1 to 200 micrograms per kilogram body weight per day, more preferably in the range of 0.1 to 100 micrograms per kilogram body weight per day, even more preferably in the range of 1.0 to 50 micrograms per kilogram body weight per day, and most preferably in the range of 1.0 to 25 micrograms per kilogram body weight per day. A pharmaceutical composition comprising the dsRNA is administered once daily, or in multiple sub-doses, for example, using sustained release formulations well known in the art. The preparation and administration of such pharmaceutical compositions may be carried out accordingly to standard techniques, as further described below.

Such dsRNA may then be used to inhibit JAK3 expression and activity in a cancer, by preparing a pharmaceutical preparation comprising a therapeutically-effective amount of such dsRNA, as described above, and administering the preparation to a human subject having a cancer expressing mutant JAK3 kinase protein, for example, via direct injection to the tumor or bone marrow. The similar inhibition of other receptor tyrosine kinases, such as VEGFR and EGFR using siRNA inhibitors has recently been described. See U.S. Patent Publication No. 20040209832, Oct. 21, 2004, McSwiggen et al.; U.S. Patent Publication No. 20030170891, Sep. 11, 2003, McSwiggen; U.S. Patent Publication No. 20040175703, Sep. 9, 2004, Kreutzer et al.

### Therapeutic Compositions: Administration.

JAK3 kinase-inhibiting therapeutic compositions useful in the practice of the methods of the invention may be administered to a mammal by any means known in the art including, but not limited to oral or peritoneal routes, including intravenous, intramuscular, intraperitoneal, subcutaneous, transdermal, airway (aerosol), rectal, vaginal and topical (including buccal and sublingual) administration.

For oral administration, a JAK3-inhibiting therapeutic will generally be provided in the form of tablets or capsules, as a powder or granules, or as an aqueous solution or suspension. Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredients is mixed with water or an oil such as peanut oil, liquid paraffin or olive oil. For intramuscular, intraperitoneal, subcutaneous and intravenous use, the pharmaceutical compositions of the invention will generally be provided in sterile aqueous solutions or suspensions, buffered to an appropriate pH and isotonicity, Suitable aqueous vehicles include Ringer's solution and isotonic sodium chloride. The carrier may consists exclusively of an aqueous buffer (“exclusively” means no auxiliary agents or encapsulating substances are present which might affect or mediate uptake of the JAK3-inhibiting therapeutic). Such substances include, for example, micellar structures, such as liposomes or capsids, as described below. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

JAK3 kinase-inhibiting therapeutic compositions may also include encapsulated formulations to protect the therapeutic (e.g. a dsRNA compound) against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075. An encapsulated formulation may comprise a viral coat protein. The viral coat protein may be derived from or associated with a virus, such as a polyoma virus, or it may be partially or entirely artificial. For example, the coat protein may be a Virus Protein 1 and/or Virus Protein 2 of the polyoma virus, or a derivative thereof.

JAK3-inhibiting compositions can also comprise a delivery vehicle, including liposomes, for administration to a subject, carriers and diluents and their salts, and/or can be present in pharmaceutically acceptable formulations. For example, methods for the delivery of nucleic acid molecules are described in Akhtar et al., 1992, Trends Cell Bio., 2, 139; DELIVERY STRATEGIES FOR ANTISENSE OLIGONUCLEOTIDE THERAPEUTICS, ed. Akbtar, 1995, Maurer et al., 1999, Mol. Membr. Biol., 16, 129-140; Hofland and Huang, 1999, Handb. Exp. Pharmacol, 137, 165-192; and Lee et al., 2000, ACS Symp. Ser., 752, 184-192. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule.

JAK3-inhibiting therapeutics can be administered to a mammalian tumor by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the therapeutic/vehicle combination is locally delivered by direct injection or by use of an inmutant pump. Direct injection of the composition, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262.

Pharmaceutically acceptable formulations of JAK3 kinase-inhibitory therapeutics include salts of the above described compounds, e.g., acid addition salts, for example, salts of hydrochloric, hydrobromic, acetic acid, and benzene sulfonic acid. A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell. For example, pharmacological compositions injected into the blood stream should be soluble. Other factors are known in the art, and include considerations such as toxicity and forms that prevent the composition or formulation from exerting its effect.

Administration routes that lead to systemic absorption (i.e. systemic absorption or accumulation of drugs in the blood stream followed by distribution throughout the entire body), are desirable and include, without limitation: intravenous, subcutaneous, intraperitoneal, inhalation, oral, intrapulmonary and intramuscular. Each of these administration routes exposes the JAK3-inhibiting therapeutic to an accessible diseased tissue or tumor. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich et al., 1999, Cell Transplant, 6, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuro-psychopharmacol Bol Psychiatry, 23, 941-949, 1999). Other non-limiting examples of delivery strategies for the JAK3-inhibiting compounds useful in the method of the invention include material described in Boado et al, 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

Therapeutic compositions comprising surface-modified liposomes containing poly (ethylene glycol) lipids (PEG-modified, or long-circulating liposomes or stealth liposomes) may also be suitably employed in the methods of the invention. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

Therapeutic compositions may include a pharmaceutically effective amount of the desired compounds in a pharmaceutically acceptable carrier or diluent. Acceptable carriers or diluents for therapeutic use are well known in the pharmaceutical art, and are described, for example, in REMINGTON'S PHARMACEUTICAL SCIENCES, Mack Publishing Co. (A. R. Gennaro edit. 1985). For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient. It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

A JAK3-inhibiting therapeutic useful in the practice of the invention may comprise a single compound as described above, or a combination of multiple compounds, whether in the same class of inhibitor (i.e. antibody inhibitor), or in different classes (i.e. antibody inhibitors and small-molecule inhibitors). Such combination of compounds may increase the overall therapeutic effect in inhibiting the progression of a mutant protein-expressing cancer. For example, the therapeutic composition may a small molecule inhibitor, such as CP-690,550 alone, or in combination with other inhibitors (e.g. WP-1034 or JAK I) targeting JAK3 activity and/or other small molecule inhibitors. The therapeutic composition may also comprise one or more non-specific chemotherapeutic agent in addition to one or more targeted inhibitors. Such combinations have recently been shown to provide a synergistic tumor killing effect in many cancers. The effectiveness of such combinations in inhibiting JAK3 activity and tumor growth in vivo can be assessed as described below.

### Identification of Mutant JAK3 Kinase-Inhibiting Compounds.

The invention also provides, in part, a method for determining whether a compound inhibits the progression of a cancer characterized by a mutant (A572V) JAK3 polynucleotide and/or polypeptide, by determining whether the compound inhibits the activity of mutant JAK3 kinase in the cancer. In some preferred embodiments, inhibition of activity of JAK3 is determined by examining a biological sample comprising cells from bone marrow, blood, or a tumor. In another preferred embodiment, inhibition of activity of JAK3 is determined using at least one mutant JAK3 polynucleotide or polypeptide-specific reagent of the invention.

The tested compound may be any type of therapeutic or composition as described above. Methods for assessing the efficacy of a compound, both in vitro and in vivo, are well established and known in the art. For example, a composition may be tested for ability to inhibit JAK3 in vitro using a cell or cell extract in which JAK3 is activated. A panel of compounds may be employed to test the specificity of the compound for JAK3 (as opposed to other targets, such as other JAK kinases).

Another technique for drug screening which may be used provides for high throughput screening of compounds having suitable binding affinity to a protein of interest, as described in published PCT application WO84/03564. In this method, as applied to mutant JAK3 polypeptides, large numbers of different small test compounds are synthesized on a solid substrate, such as plastic pins or some other surface. The test compounds are reacted with mutant JAK3 polypeptide, or fragments thereof, and washed. Bound mutant polypeptide (e.g. mutant JAK3 kinase polypeptide) is then detected by methods well known in the art. Purified mutant JAK3 polypeptide can also be coated directly onto plates for use in the aforementioned drug screening techniques. Alternatively, non-neutralizing antibodies can be used to capture the peptide and immobilize it on a solid support.

A compound found to be an effective inhibitor of JAK3 activity in vitro may then be examined for its ability to inhibit the progression of a cancer expressing mutant JAK3 kinase polypeptide, in vivo, using, for example, mammalian bone marrow transplants (e.g. mice) harboring human leukemias that are driven by the mutant JAK3 protein. In this procedure, bone marrow cells known to be driven by mutant JAK3 kinase (e.g. CMK cells) are transplanted in the mouse. The growth of the cancerous cells may be monitored. The mouse may then be treated with the drug, and the effect of the drug treatment on cancer phenotype or progression be externally observed. The mouse is then sacrificed and the transplanted bone marrow removed for analysis by, etc., IHC and Western blot. Similarly, mammalian xenografts may be prepared, by standard methods, to examine drug response in solid tumors expressing a mutant JAK3 kinase. In this way, the effects of the drug may be observed in a biological setting most closely resembling a patient. The drug's ability to alter signaling in the cancerous cells or surrounding stromal cells may be determined by analysis with phosphorylation-specific antibodies. The drug's effectiveness in inducing cell death or inhibition of cell proliferation may also be observed by analysis with apoptosis specific markers such as cleaved caspase 3 and cleaved PARP.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

The teachings of all references cited above and below are hereby incorporated herein by reference. The following Examples are provided only to further illustrate the invention, and are not intended to limit its scope, except as provided in the claims appended hereto. The present invention encompasses modifications and variations of the methods taught herein which would be obvious to one of ordinary skill in the art.

### Example 1

### An AML Cell Line Lacking FLT3 or KIT Mutations But Possessing Activated STAT5

Immunoblot analysis using a phospho-STAT5 antibody was used to screen several human AML cell lines for constitutive phosphorylation of STAT5. Antibodies for immunoblotting were anti-phospho-STAT5 (Cell Signaling Technology, Beverly, Mass.), anti-STAT5, anti-JAK1 and anti-TYK2 (BD Transduction Laboratories, BD Pharmingen, San Diego, Calif.), anti-JAK2, anti-JAK3, anti-phospho-JAK1, anti-phospho-JAK3 (Santa Cruz Biotechnology, Inc., Santa Cruz, Calif.).

AML-193, GDM-1, CMK, K562 and Ba/F3 cells were obtained from the German National Resource Centre for Biological Material (DSMZ). GF-D8 cells were generously provided by Dr. Orietta Spinelli, Laboratorio Paolo Belli, Bergamo, Italy. All cell lines were maintained in RPMI 1640 supplemented with 10% FCS, 1 unit/mL penicillin G, and 1 μg/mL streptomycin at 37° C. and 5% CO2. Western blotting was carried out according to standard methods, following the Western Blotting Protocol (Cell Signaling Technology, Inc., 2005-2006 Catalogue).

STAT5 was found to be constitutively activated in CMK cells (FIG. 2). CMK cells are a megakaryoblastic cell line derived from a patient with Down syndrome and M7 leukemia. Activating FLT3 or KIT mutations are commonly responsible for constitutive activation of STAT5 in AML. Accordingly, mutation analysis was carried out as previously described (see Goemans et al., Leukemia 19:1536-42 (2005); O'Farrell et al., Clin. Cancer Res. 9: 5465-76 (2003) to determine whether any FLT3 or KIT mutations exist in the CMK cell line. Denaturing-HPLC analysis revealed that the CMK cell line does not possess activating FLT3 or KIT mutations.

### Example 2

### Identification of JAK3 Kinase Activity in an AML Cell Line by Global Phosphopeptide Profiling

The global phosphorylation profile of kinase activation in the CMK AML cell line was examined using a recently described and powerful technique for the isolation and mass spectrometric characterization of modified peptides from complex mixtures (the “IAP” technique, see Rush et al., supra). The IAP technique was performed using a phosphotyrosine-specific antibody (CELL SIGNALING TECHNOLOGY, INC., Beverly, Mass., 2003/04 Cat. #9411) to isolate, and subsequently characterize, phosphotyrosine-containing peptides from extracts of the CMK cell lines.

Specifically, the IAP approach was employed to facilitate the identification of tyrosine kinases responsible for STAT5 phosphorylation in the CMK cell line. STAT5 is a member of the STAT family of transcription factors. The activated tyrosine kinases typically phosphorylate one or more signal transducer and activator (STAT) of transcription factors, which translocate to the cell nucleus and regulate the expression of genes associated with survival and proliferation. The phosphorylation and activation of STAT family members has been previously been described in a wide range of human leukemias. In addition, animal models have demonstrated the important role of STAT in leukemogenesis. STAT5 has been found to be constitutively tyrosine phosphorylated in about 70% of patients with AML. Activating mutations of FLT3 or KIT can account for up to 35% of patients with STAT5 phosphorylation. However, a significant percentage of patients lacking these mutations maintain phosphorylation of STAT5. Hence, it was hypothesized that the upstream activator of STAT5 in some of these patients is an activated tyrosine kinase, and activation of these kinases was examined.

### Cell Culture.

CMK cells were maintained in RPMI 1640 supplemented with 10% FCS, I unit/mL penicillin G, and 1 μg/mL streptomycin at 37° C. and 5% CO2.

Phosphopeptide Immunoprecipitation.

2×10⁸ cells were lysed in urea lysis buffer (20 mM HEPES pH 8.0, 9M urea, 1 mM sodium vanadate, 2.5 mM sodium pyrophosphate, 1 mM beta-glycerophosphate) at 1.25×10³ cells/ml and sonicated. Sonicated lysates were cleared by centrifugation at 10,000 RPM, and proteins reduced and alkylated as described previously⁵. Samples were diluted with 20 mM HEPES pH 8.0 to a final urea concentration of 2M. Soluble trypsin (1 mg/ml in 0.001 M HCl) or elastase (2 mg/ml in H₂O) was added to the clarified lysate at a 1:100 dilution.

Samples were digested overnight at room temperature. Following digestion, lysates were acidified to a final concentration of 1% TFA. Peptide purification was carried out using Sep-Pak C₁₈ columns. Following purification, all elutions (8%, 12%, 15%, 18%, 22%, 25%, 30%, 35% and 40% acetonitrile in 0.1% TFA) were combined into one fraction for analysis. Dried peptides were resuspended in 1.4 ml of MOPS immunoprecipitation buffer (50 mM MOPS/NaOH pH 7.2, 10 mM Na₂HPO₄, 50 mM NaCl) and insoluble material removed by centrifugation at 3000 RPM for ten minutes.

The phosphotyrosine monoclonal antibody P-Tyr-100 from ascites fluid was coupled non-covalently to protein G-Agarose (Roche) at 4 mg/ml beads overnight at 4° C. After coupling, antibody-resin was washed twice with PBS and three times with MOPS immunoprecipitation buffer (5-10 bead volumes of buffer for each wash). Immobilized antibody (40 μl, 160 μg) was added as a 1:1 slurry in MOPS immunoprecipitation buffer to the solubilized peptide fraction, and the mixture was incubated overnight at 4° C. The immobilized antibody beads were washed three times with 1 ml

MOPS immunoprecipitation buffer and twice with 1 ml water, all at 4° C. Peptides were eluted from beads by incubation with 60 μl of 0.1% TFA followed by a second elution with 40 μl of 0.1% TFA.

### Analysis by LC-MS/MS Mass Spectrometry.

Peptides in the immunoprecipitation eluate (53 μl) were concentrated and separated from eluted antibody using ZipTip μC18 columns (Millipore). Peptides were eluted from the microcolumns with 1 μl of 60% MeCN, 0.1% TFA into 7.6 μl of 0.4% acetic acid/0.005% heptafluorobutyric acid (HFBA). The sample was loaded onto a 10 cm×75 μm PicoFrit capillary column (New Objective) packed with Magic C18 AQ reversed-phase resin (Michrom Bioresources) using a Famos autosampler with an inert sample injection valve (Dionex). The column was developed with a 45-min linear gradient of acetonitrile in 0.4% acetic acid, 0.005% HFBA delivered at 280 nm in (Ultimate, Dionex).

Tandem mass spectra were collected in a data-dependent manner with an LCQ Deca XP Plus ion trap mass spectrometer (ThermoFinnigan), using a top-four method, a dynamic exclusion repeat count of 1, and a repeat duration of 0.5 min.

### Database Analysis & Assignments.

MS/MS spectra were evaluated using TurboSequest (ThermoFinnigan) (in the Sequest Browser package (v. 27, rev. 12) supplied as part of BioWorks 3.0). Individual MS/MS spectra were extracted from the raw data file using the Sequest Browser program CreateDta, with the following settings: bottom MW, 700; top MW, 4,500; minimum number of ions, 20; minimum TIC, 4×10⁵ and precursor charge state, unspecified. Spectra were extracted from the beginning of the raw data file before sample injection to the end of the eluting gradient. The IonQuest and VuDta programs were not used to further select MS/MS spectra for Sequest analysis. MS/MS spectra were evaluated with the following TurboSequest parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

TurboSequest (ThermoFinnigan) searches were done against the NCBI human database released on Feb. 23, 2004 containing 27,175 proteins allowing for oxidized methionine (M+16) and phosphorylation (Y+80) as dynamic modifications.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell. Proteomics 3: 531-533 (2004)), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site.

For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. Assigned sequences were accepted or rejected following a conservative, two-step process. In the first step, a subset of high-scoring sequence assignments was selected by filtering for XCorr values of at least 1.5 for a charge state of +1, 2.2 for +2, and 3.3 for +3, allowing a maximum RSp value of 10. Assignments in this subset were rejected if any of the following criteria were satisfied: (i) the spectrum contained at least one major peak (at least 10% as intense as the most intense ion in the spectrum) that could not be mapped to the assigned sequence as an a, b, or y ion, as an ion arising from neutral-loss of water or ammonia from a b or y ion, or as a multiply protonated ion; (ii) the spectrum did not contain a series of b or y ions equivalent to at least six uninterrupted residues; or (iii) the sequence was not observed at least five times in all the studies we have conducted (except for overlapping sequences due to incomplete proteolysis or use of proteases other than trypsin). In the second step, assignments with below-threshold scores were accepted if the low-scoring spectrum showed a high degree of similarity to a high-scoring spectrum collected in another study, which simulates a true reference library-searching strategy. All spectra supporting the final list of assigned sequences (not shown here) were reviewed by at least three scientists to establish their credibility.

The foregoing IAP analysis identified 94 non-redundant phosphotyrosine-containing peptides, 68 phosphotyrosine sites, and 56 tyrosine phosphorylated proteins, the majority of which are novel, from CMK cells (data not shown). Among the tyrosine-phosphorylated kinases detected, we found JAK3 and Tyk2 (unpublished data).

### Example 3

### Growth Inhibition of JAK3-Expressing AML Cell Line Using a Pan-JAK Kinase Inhibitor and siRNA

In order to determine whether the detected JAK3 kinase is driving cell growth and survival in the CMK AML cell line, the ability of both a pan-JAK inhibitor, JAK I, and siRNA to inhibit growth of these cells was examined.

### JAK I Inhibition.

JAK I (Calbiochem, San Diego, Calif.) is a potent inhibitor of the JAK family of kinases, including JAK3. To confirm that JAK3 is driving the proliferation of CMK cells, the effect of JAK I on the growth and/or viability of CMK cells was examined. CMK and K562 cells (as a control) were treated for seventy hours with increasing doses of JAK I inhibitor up to 2 micrograms/ml. The number of viable cells was determined with the CellTiter 96 AQᵤₑₒᵤₛ One solution cell proliferation assay (Promega). Apoptosis was measured with a fluorescent stain measuring annexin V.

Treatment of CMK cells with JAK I resulted in inhibition of cell proliferation and a significant decrease in viability (FIGS. 3A and 3B). The JAK I inhibitor had no significant effect on the growth and viability of the control cell line, K562.

To further confirm the effect of the JAK I on the CMK cell line, Western blot analysis (as described above) was performed following exposure to a range of inhibitor concentrations, and the activity of the JAK3 downstream target, STAT5 examined (see FIG. 3C). The results indicate that inhibition of JAK kinases in the CMK cell line results in inhibited phosphorylation of its downstream target STAT5 as well.

siRNA Inhibition.

To determine which JAK family member or members may contribute to the growth and viability of the CMK cells, the expression of individual JAK family members was down regulated with siRNA.

JAK1, JAK2, JAK3 and TYK2 SMARTpool® siRNA duplexes (proprietary target sequences) were purchased from Dharmacon Research, Inc. (Lafayette, Colo.). A non-specific SMARTpool® siRNA was used as a control. Cells were transfected with the various siRNA via electroporation. Briefly, cells were pulsed once (CMK 20 ms; 250V, K562 20 ms; 285V) using a square-wave electroporator (BTX Genetronics, San Diego, Calif.), incubated at room temperature for 30 min and transferred to 96 well, 24 well and/or 6 well plates (Becton Dickinson, Franklin Lakes, N.J.). Western blotting was carried out as described above

As shown in FIG. 4A, immunoblot analysis revealed that the expression of JAK1, JAK2, JAK3 and TYK2 was specifically and significantly reduced by 89%, 92%, 95%, and 93% at 48 hours following transfection of the respective JAK family member siRNA into CMK cells. Non-specific SiRNA had no effect on the expression of any of the JAK family members. Interestingly, down regulation of either JAK1 or JAK3 resulted in inhibition of cell growth (FIG. 4B) and these findings were statistically significant and reproducible. Moreover, treatment with either JAK1 or JAK3 siRNA resulted in a decrease in the viability of the CMK cell line (FIG. 4B). The effectiveness of the JAK1 and JAK3 siRNAs is shown in FIG. 4C by measuring the level of the apoptosis marker, annexin V. As shown in FIG. 4D, down regulation of either JAK1 or JAK3, also resulted in a significant decrease in the tyrosine phosphorylation of STAT5.

These results indicate that either or both of JAK3 kinase and/or JAK1 kinase are driving the proliferation and growth of these AML cells, but that such growth and proliferation may be inhibited by using siRNA to inhibit JAK3 kinase (or JAK1 kinase) expression.

### Example 4

### siRNA-Induced Down-Regulation of JAK3 and JAK1 Cooperatively Inhibits Growth and Survival of an AML Cell Line

Since a decrease in growth, viability and the phosphorylation of STAT5 was observed when either JAK1 or JAK3 was down regulated using siRNA, the effect of down regulating both kinases simultaneously was examined. siRNA were prepared and employed substantially as described in Example 3 above.

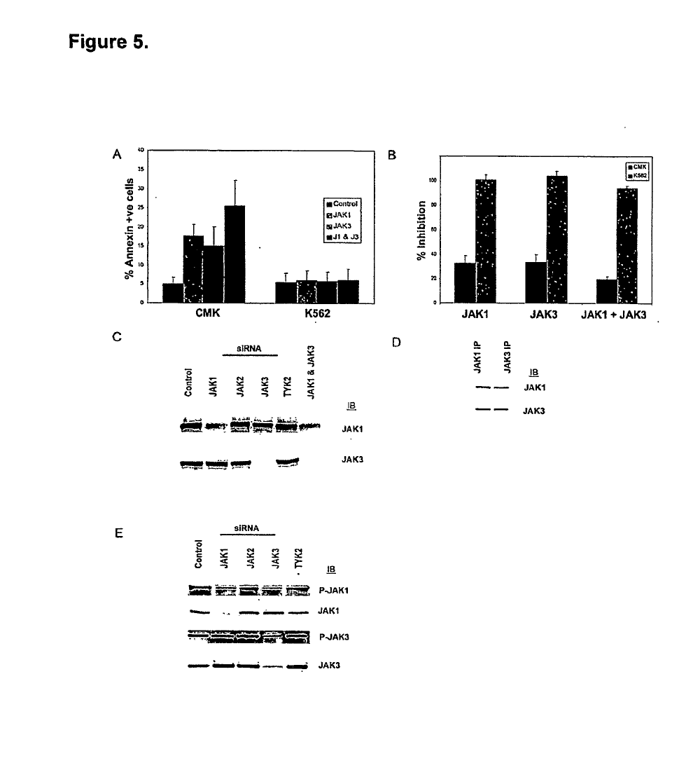

The results indicate that concurrent siRNA-induced down regulation of JAK1 and JAK3 results in a greater reduction of cell growth and cell viability as compared to down regulation of JAK1 and JAK3 alone (FIGS. 5A, 5B and 5C). Accordingly, it was concluded that both JAK3 and JAK1 must play an important role in the growth and survival of the CMK cells.

### Example 5

### Identification of Mutant Form of JAK3 Kinase

Since both JAK1 and JAK3 expression are required for the growth and viability of CMK cells and for phosphorylation of STAT5, both kinases were examined for the presence of activating mutations in the kinase domain.

Primers for the kinase domain and JH2 pseudokinase domain of JAK3 were constructed as follows:

Forward:

(SEQ ID NO: 3)

5′-GTAAAACGACGGCCAGTGGTGTTCCCCTAAGGCAGGT-3′,
Reverse:

(SEQ ID NO: 4)

5′-CAGGAAACAGCTATGACCGTGTTGCTGGGGCTGTCATA-3′.

Bidirectional sequence traces were assembled and analyzed for mutations using Mutation Surveyor version 2.5 (SoftGenetics, State College, Pa.).

### PCR Assay

Genomic DNA was purified from CMK cells. The JAK3 kinase and JH2 pseudokinase region were amplified with the use of primer pairs specific to JAK3 flanking the appropriate exons.

Interestingly, no mutations were found following the sequencing of the kinase regions of either JAK3 or JAK1. Since, Levine et al. (2005), supra., had recently reported detection of an activating mutation in the JAK homology 2 (JH2) pseudokinase domain of JAK2 in patients with polycythemia vera (PV), the JH2 domain of both JAK1 and JAK3 in the CMK cell line were also sequenced. No mutation was detected in the JH2 region of JAK1, however, a novel mutation (A572V) was detected in the JH2 domain of JAK3. Of note, the A572V mutation of JAK3 is not in the homologous location to the previously identified JAK2 mutations.

### Example 6

### Inhibition of Mutant JAK3 Kinase by siRNA Inhibits JAK1 and STAT5 Phosphorylation

Although a novel mutation was detected in the JH2 region of JAK3 but no mutations were detected in JAK1, the above-described experiments indicated that both JAK3 and JAK1 play an important role in the survival of the CMK cells. Since it had previously been reported that JAK1 is a superior activator of STAT5, while JAK3 is a superior activator of JAK1 (see Saharinen et al., supra.), the interaction of mutant (A572V) JAK3 and JAK1, and the activation of STAT5 by JAK1 were further examined.

Cell extracts were prepared from CMK cells substantially as described above in Example 1. Co-immunoprecipitation experiments were carried out, as described in the Cell Signaling Technology Catalog 2005-2006.

Co-immunoprecipitation results demonstrated that JAK1 co-precipitates with JAK3 and vice versa (see FIG. 5D). Accordingly, the activation status of JAK1 and JAK3 following siRNA-induced down regulation of these kinases was then examined. siRNA were prepared and employed substantially as described in Example 3 above.

As shown in FIG. 5E, siRNA-induced down regulation of JAK3 results in a decrease in phosphorylation of both JAK3 and JAK1 while siRNA-induced down regulation of JAK1 only decreases expression and phosphorylation of JAK1. siRNA down regulation of JAK1 decreases phosphorylation of STAT5 (FIG. 4D). Taken together, these data are consistent with mutant (572V) JAK3 activating JAK1, and JAK1 in turn being responsible for directly phosphorylating STAT5 in this AML cell line.

### Example 7

### Mutant JAK3 Kinase Drives Growth and Survival of Transformed Mammalian Cell Line

To assess the whether the A572V JAK3 mutation can transform normal cells into a cancerous phenotype, Ba/F3 cells were transfected with either wild-type JAK3 or mutant (A572V) JAK3.

CMK cells were cultured as described above in Example 1. A cDNA encoding the S isoform of human JAK3 was prepared from a purchased clone encoding the M isoform of human JAK 3 (Origene, Rockville, Md.). The required C-terminal region for the S isoform of JAK3 was amplified by RT-PCR using cDNA from the CMK cell line, then directionally inserted as a 1.0 kb SrfI-XbaI fragment. Following transfer of the resulting JAK3 (S isoform) cDNA to pRC/CMV as a 4.7 kb NotI fragment, the A572V mutation was introduced using the GeneTailor™ site-directed mutagenesis system (Invitrogen, Carlsbad, Calif.). Full-length sequencing confirmed the identities of the cDNAs encoding JAK3(WT) and JAK3(A572V).

Primer sequences are as follows:

RT-PCR:

(SEQ ID NO: 5)

Forward: 5′-CGCTAGGCGACAATACAGGT-3′

(SEQ ID NO: 6)

Reverse: 5′-ATAATTCTAGACACATATGCCCATCTG-3′
GeneTailor ™(JAK3(A572V)):

(SEQ ID NO: 7)

Forward: 5′-GCATGGAGTCATTCCTGGAAGTAGCGAGCTTG-3′

(SEQ ID NO: 8)

Reverse: 5′-CTTCCAGGAATGACTCCATGCAGTTCTTGTG-3′.

Ba/F3 cells were transfected with either WT JAK3 or A572V JAK3 via electroporation (20 ms; 285V) using a square-wave electroporator (BTX Genetronics, San Diego, Calif.). 24 hours following transfection, 750 g/ml G418 was added in order to begin the selection of stable transfectants. Stable transfectants were plated in 96 well plates with various concentrations of murine IL-3 and growth was measured 5 days later using MTS reagent. Western blot experiments were carried out substantially as described in Example 1 above.

As shown in FIG. 6A, STAT5 was found to be constitutively tyrosine phosphorylated in cells transfected with A572V JAK3 but not in cells transfected with wild-type JAK3. The ability of these transfected cell lines to grow in a range of IL-3 concentrations was then determined and compared to the growth of untransfected Ba/F3 cells. As shown in FIG. 6B, transfection of A572V JAK3 was not able to completely render the Ba/F3 cells IL-3 independent. However transfection of A572V JAK3 did confer a significant growth advantage, manifested as increased sensitivity to IL-3 as compared to the growth of either the wild-type JAK3 transfected cells or the untransfected Ba/F3 cells.

### Example 8

### Examination of Mutant JAK3 Kinase Expression in Human Leukemias and Myeloproliferative Disorders

Since the A572V JAK3 mutation was shown to play a significant role in the growth and survival of the AML cell line (CMK) and that this mutation confers a growth advantage to Ba/F3 cells, the prevalence of the A572V JAK3 mutation in a variety of hematological malignancies was examined.

The following were tissues were examined for the presence of the A572V JAK3 activating mutation: 30 AML cell lines, 222 AML patient samples of various FAB classifications, 30 chronic myelomonocytic leukemia samples, and samples from patients with myeloproliferative disorders, including PV and essential thrombocythemia. Because the CMK cell line was originally classified as an AML-M7 from a patient with Down syndrome (DS), 3 Down syndrome M7 and 3 non-Down syndrome M7 samples were also examined for the presence of the A572V JAK3 activating mutation. Patient genomic DNA was purified and screened using PCR to assess mutational status of the JAK3 gene.

Although, the mutation was not detected in any of the patient samples tested or any other AML cell lines besides the CMK cell line, it is anticipated that it will be detected in a subset of AML patients in a wider population of samples.

### Example 9

### Detection of Mutant JAK3 Kinase Expression in a Human Cancer Sample Using PCR Assay

The presence of mutant (A572V) JAK3 kinase protein in a human cancer sample may be detected using either genomic or reverse transcriptase (RT) polymerase chain reaction (PCR), as previously described. See, e.g., Cools et al., N. Engl. J. Med. 348:1201-1214 (2003).

Briefly and by way of example, bone marrow samples may be obtained from a patient having AML using standard techniques. PCR probes against truncated JAK3 kinase or mutant JAK3 kinase protein are constructed. RNeasy Mini Kit (Qiagen) may be used to extract RNA from human bone marrow samples. DNA may be extracted with the use of DNeasy Tissue Kit (Qiagen). For RT-PCR, first-strand cDNA is synthesized from, e.g., 2.5 μg of total RNA with the use, for example, of SuperScript™ III first-strand synthesis system (Invitrogen) with oligo (dT)₂₀. Then, the mutant JAK3 kinase gene is amplified with the use of primer pairs, e.g. A572V JAK3 forward and reverse primers (see Example 7 above). For genomic PCR, amplification of the mutant gene may be performed with the use of Platinum Taq DNA polymerase high fidelity (Invitrogen) with primer pairs, e.g. A572V JAK3 forward and reverse primers (see Example 7 above).

Such an analysis will identify a patient having a cancer characterized by expression of the mutant (A572V) JAK3 kinase protein, which patient is a candidate for treatment using a JAK3-inhibiting therapeutic, such as CP-690,550.

1. An isolated polynucleotide comprising a nucleotide sequence at least 95% identical to a sequence selected from the group consisting of:
(a) a nucleotide sequence encoding a mutant Janus Kinase-3 (JAK3) polypeptide comprising the amino acid sequence of SEQ ID NO: 1 but having an alanine-to-valine point mutation at residue 572;
(b) a nucleotide sequence encoding a mutant JAK3 kinase polypeptide, the nucleotide sequence comprising the nucleotide sequence of SEQ ID NO: 2 but having an alanine-to-valine codon mutation at nucleotides 1773-1775;
(c) a nucleotide sequence encoding a mutant JAK3 kinase polypeptide comprising the kinase domain (residues 822-1095 of SEQ ID NO: 1) and JH2-pseudokinase domain (residues 521-777 of SEQ ID NO: 1) of JAK3 kinase, but having an alanine-to-valine point mutation at residue 572 in the JH2-pseudokinase domain;
(d) a nucleotide sequence encoding a mutant JAK3 kinase polypeptide, the nucleotide sequence comprising the nucleotide sequences encoding the kinase domain of JAK3 (nucleotides 2523-3342 of SEQ ID NO: 2) and the JH2-pseudokinase domain of JAK3 (nucleotides 1620-2388 of JAK3), but having an alanine-to-valine codon mutation at nucleotides 1773-1775 in the JH2-pseudokinase domain;
(e) a mutant JAK3 kinase nucleotide sequence comprising at least six contiguous nucleotides encompassing an alanine-to-valine codon mutation at nucleotides 1773-1775 of SEQ ID NO: 2;
(f) a nucleotide sequence encoding a mutant JAK3 polypeptide comprising at least six contiguous amino acids encompassing an alanine-to-valine point mutation at residue 572 of SEQ ID NO: 1; and
(g) a nucleotide sequence complementary to any of the nucleotide sequences of (a)-(f).

2. An isolated polypeptide encoded by the polynucleotide of claim 1.

3. An isolated polynucleotide that hybridizes under stringent hybridization conditions to the isolated polynucleotide of claim 1, wherein the isolated polynucleotide that hybridizes does not hybridize under stringent hybridization conditions to a polynucleotide having a nucleotide sequence consisting of only A residues or of only T residues.

4. The isolated polynucleotide of claim 3, wherein the isolated polynucleotide further comprises a detectable label.

5. A recombinant vector comprising the isolated nucleic acid molecule of claim 1.

6. (canceled)

7. A recombinant host cell comprising the recombinant vector of claim 5.

8.-9. (canceled)

10. An isolated polypeptide comprising an amino acid sequence at least 95% identical to a sequence selected from the group consisting of:
(a) an amino acid sequence encoding a mutant Janus Kinase-3 (JAK3) polypeptide comprising the amino acid sequence of SEQ ID NO: 1, but having an alanine-to-valine point mutation at residue 572;
(b) an amino acid sequence encoding a mutant JAK3 kinase polypeptide comprising the kinase domain (residues 822-1095 of SEQ ID NO: 1) and the JH2 pseudo-kinase domain (residues 521-777 of SEQ ID NO: 1), but having an alanine-to-valine point mutation at residue 572 in the JH2 pseudo-kinase domain; and
(c) an amino acid sequence encoding a mutant JAK3 polypeptide comprising at least six contiguous amino acids encompassing residue 572 of SEQ ID NO: 1, but having an alanine-to-valine point mutation at this position.

11.-12. (canceled)

13. An isolated reagent that specifically binds to or detects a mutant JAK3 kinase polypeptide of claim 10, but does not bind to or detect wild type JAK3.

14. The isolated reagent of claim 13, wherein the reagent is an antibody or a heavy-isotope labeled (AQUA) peptide.

15. The heavy isotope labeled (AQUA) peptide of claim 14, wherein the peptide comprises four or more amino acids encompassing the alanine-to-valine mutation at residue 572 in the JH2-pseudokinase domain of JAK3 kinase (SEQ ID NO: 2).

16. A kit for the detection of mutant (A572V) JAK3 kinase in a biological sample, the kit comprising the isolated reagent of claim 13 and one or more secondary reagents.

17. A method for detecting the presence of a mutant JAK3 polynucleotide and/or polypeptide in a cancer, the method comprising:
(a) obtaining a biological sample from a subject having cancer; and
(b) utilizing at least one reagent that detects a polynucleotide of claim 1 or a polypeptide encoded by the polynucleotide of claim 1 to determine whether a mutant (A572V) JAK3 polynucleotide and/or polypeptide is present in the biological sample.

18. The method of claim 17, wherein the cancer is leukemia.

19. The method of claim 18, wherein the leukemia is acute myelogenous leukemia (AML).

20. The method of claim 17, wherein the presence of a mutant (A572V) JAK3 polynucleotide or polypeptide identifies a cancer that is likely to respond to a composition comprising at least one JAK3 kinase-inhibiting therapeutic.

21. The method of claim 20, wherein the JAK3 kinase-inhibiting therapeutic is CP-690,550 or its analogues.

22. The method of claim 20, wherein the JAK3 kinase-inhibiting therapeutic is WP-1034 or its analogues.

23. The method of claim 17, wherein the method is implemented in a flow-cytometry (FC), immuno-histochemistry (IHC), or immuno-fluorescence (IF) assay format.

24. The method of claim 17, wherein the method is implemented in a polymerase chain reaction (PCR) sequencing assay format.

25. The method of claim 17, wherein the activity of the mutant JAK3 kinase polypeptide is detected.

26. A method for determining whether a compound inhibits the progression of a cancer characterized by a mutant (A572V) JAK3 kinase polynucleotide and/or polypeptide, the method comprising determining whether the compound inhibits the expression and/or activity of the mutant (A572V) JAK3 kinase polypeptide in the cancer.

27. The method of claim 26, wherein inhibition of expression and/or activity of the mutant JAK3 kinase polypeptide is determined using at least one reagent that detects a polynucleotide of claim 1.

28. A method for inhibiting the progression of a cancer that expresses a mutant (A572V) JAK3 kinase polypeptide, the method comprising the step of inhibiting the expression and/or activity of the mutant JAK3 kinase polypeptide in the cancer.

29. The method of claim 28, wherein the cancer is leukemia.

30. The method of claim 29, wherein the leukemia is acute myelogenous leukemia (AML).

31. The method of claim 28, wherein expression and/or activity of mutant JAK3 kinase is inhibited with a composition comprising CP-690,550 and/or its analogues.

32. The method of claim 28, wherein the expression and/or activity of mutant JAK3 kinase is inhibited with a composition comprising WP-1034 and/or its analogues.
